(19) United States
(12) Patent Application Publication
(10) Pub. No.: US 2026/0250428 A1
(43) Pub. Date: Aug. 27, 2026

(54) MODIFIED CARBOXYMETHYL CELLULOSE, THEIR COMPOSITIONS AND THE USES THEREOF

(71) Applicant: ISP Investments LLC, Wilmington, DE (US)

(72) Inventors: Gijsbert KROON, Giessenburg (NL); Sounak SARKAR, Hillsborough, NJ (US); David Hutchings HOWE, Wilmington, DE (US); Solomon Howard JACOBSON, Berkeley Heights, NJ (US); Helen K. CHOWANIEC, Westfield, NJ (US); Seher OZKAN, Clifton, NJ (US); John Aaron BULL, IV, Wilmington, DE (US); Petros GEBRESELASSIE, Whitehouse Station, NJ (US); Hani M. FARES, Somerset, NJ (US)

(73) Assignee: ISP Investments LLC, Wilmington, DE (US)

(21) Appl. No.: 18/854,507

(22) PCT Filed: Jan. 31, 2023
(86) PCT No.: PCT/US2023/011942

§ 371 Date: Oct. 4, 2024

Related U.S. Application Data
(60) Provisional application No. 63/327,465, filed on Apr. 5, 2022.

(51) Int. Cl. C08B 15/04; A61K 6/35; A61K 8/73; A61Q 11/00

(52) U.S. Cl. CPC C08B 15/04; A61K 6/35; A61K 8/731; A61Q 11/00; A61K 2800/48

(57) ABSTRACT

Disclosed is a modified carboxylated polysaccharide, more particularly a modified carboxymethyl cellulose (CMC) functionalized with at least one mono-valent (M⁺) cation and at least one di-valent (M⁺⁺) cation. Further disclosed herein is a mucoadhesive composition comprising the modified carboxymethyl cellulose (CMC) functionalized with at least one mono-valent (M⁺) cation and at least one di-valent (M⁺⁺) cation. Further disclosed is application of the mucoadhesive composition in denture adhesives and toothpaste compositions.

### FIELD OF THE INVENTION

The present application relates to modified carboxylated polysaccharides, particularly to a modified carboxymethyl cellulose (CMC), and more particularly, to a water-soluble salt of carboxymethyl cellulose (CMC) functionalized with at least one mono-valent (M⁺) cation and at least one di-valent (M⁺⁺) cation, a process for preparing the same, and applications thereof.

### BACKGROUND OF THE INVENTION

Carboxylated Polysaccharides, such as carboxymethyl cellulose (CMC), carboxymethyl inulin, carboxymethyl guar or carboxymethyl starch are long-chain polymers with their solution characteristics depending on the average chain length, degree of polymerization (DP), and the degree of substitution (DS). The average amount of hydroxyl group that is substituted with sodium carboxyethyl ether groups in the cellulose structure at C2, C3 and C6 is represented by DS values. As molecular weight increases, the viscosities of CMC solutions increase. Sodium carboxymethylcellulose (NaCMC) is an anionic, water soluble polyelectrolyte with numerous applications in the food, pharmaceutical, personal care, home care, cosmetics, paper, and other industries. Sodium CMC is inherently or readily biodegradable at DS values of 0.7 and below. As the carboxymethyl groups on CMC are substituted with cations other than sodium, such as divalent cations like calcium, magnesium, zinc, tin, etc., various property changes including but not limited to decreased viscosity and reduced water solubility are typically observed.

“Effect of mono- and di-valent salts on the properties of carboxymethyl cellulose hydrogel under irradiation technique” published in International Journal of Chemical Sciences, 627-634, 10 (2), January 2012-discloses a study of Swelling properties and gel fraction of CMC hydrogels prepared by gamma radiation in presence of mono- and di-valent salts (NaCl and CaCl₂)).

US Patent Publication US2005/0032940 (assigned to HealthTech Corporation) discloses an adhesion-type denture adhesive comprising water soluble polymer containing 0.1 to 20% by weight of calcium sulfate and 3 to 60% by weight of sodium carboxymethyl cellulose for forming a gel through reaction with the calcium sulfate upon contact with an aqueous component.

The present inventors disclose herein a high molecular weight modified polysaccharides, particularly a modified carboxymethyl cellulose, wherein the said carboxymethyl cellulose is functionalized with at least one mono-valent cation (M⁺) and at least one di-valent cation (M⁺⁺), without compromising on the water solubility and biodegradable properties of the substituted cellulose.

### SUMMARY OF THE INVENTION

The primary aspect of the present application is to provide a modified carboxylated polysaccharide, particularly a modified carboxymethyl cellulose (CMC), wherein the modified carboxymethyl cellulose (CMC) is a water-soluble salt of carboxy methyl cellulose (CMC) functionalized with at least one mono-valent cation (M⁺) and at least one di-valent (M⁺⁺) cation.

In another aspect, the present application provides a functionalized carboxymethyl cellulose (CMC) having a structure of (I):

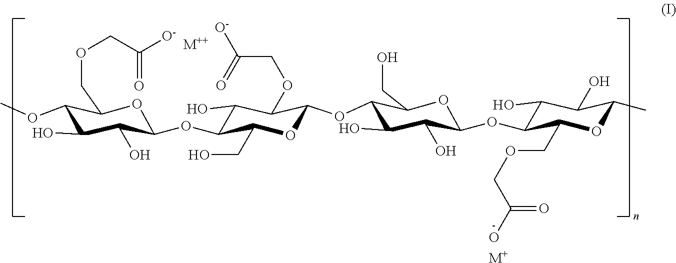

wherein M⁺ is a mono-valent cation selected from the group consisting of sodium, potassium, and lithium; and M⁺⁺ is a di-valent cation selected from the group consisting of calcium, magnesium, zinc, tin, copper, and combinations thereof; and wherein n=2 to 500, M⁺=0.1 to 10 wt. % and M⁺⁺=0.1 to 10 wt. % of the carboxymethyl cellulose.

In another aspect, the functionalized carboxymethyl cellulose is selected from the group comprising: (i) sodium-calcium carboxymethyl cellulose (Na⁺—Ca⁺⁺-CMC), (ii) sodium-magnesium carboxymethyl cellulose (Na⁺-Mg⁺⁺-CMC), (iii) sodium-zinc carboxymethyl cellulose (Na⁺—Zn⁺⁺-CMC), (iv) sodium-calcium-zinc carboxymethyl cellulose (Na⁺—Ca⁺⁺—Zn⁺⁺-CMC), (v) sodium-calcium-magnesium carboxymethyl cellulose (Na⁺-Ca⁺⁺-Mg⁺⁺-CMC), (vi) sodium-magnesium-zinc-carboxymethyl cellulose (Na⁺-Mg⁺⁺—Zn⁺⁺-CMC), (vii) sodium-tin-carboxy methyl cellulose (Na⁺—Sn⁺⁺-CMC), (viii) sodium-calcium-tin-carboxymethyl cellulose (Na⁺—Ca⁺⁺—Sn⁺⁺-CMC), (ix) sodium-magnesium-tin-carboxymethyl cellulose (Na⁺-Mg⁺⁺—Sn⁺⁺-CMC), or (x) sodium-zinc-tin-carboxymethyl cellulose (Na⁺—Zn⁺⁺—Sn⁺⁺-CMC).

In another aspect, the present application provides a mucoadhesive composition comprising: (i) (a) 0.1 wt. % to 60 wt. % of water soluble salt of carboxymethyl cellulose (CMC) functionalized with at least one mono-valent (M⁺) cation and at least one di-valent (M⁺⁺) cation; or (b) 0.1 wt. % to 60 wt. % of a combination of (b1) water soluble salt of carboxymethyl cellulose (CMC) functionalized with at least one mono-valent (M⁺) cation and at least one di-valent (M⁺⁺) cation, and (b2) water soluble salt of carboxymethyl cellulose (CMC) functionalized with at least one mono-valent (M⁺) cation, (ii) 0.1 wt. % to 60 wt. % of a maleic acid or maleic anhydride co-polymer of C1 to C4 alkyl vinyl ether and maleic acid or anhydride monomers in substantially alternating monomer structure and having 50 to 90 wt. % of the acid moieties in the polymer converted to a metal salt(s) or mixed salts; and (iii) 0.1 wt. % to 80 wt. % of at least one additional ingredient.

In another aspect, the present application provides a denture adhesive composition comprising: (i) (a) 0.1 wt. % to 60 wt. % of water soluble salt of carboxymethyl cellulose (CMC) functionalized with at least one mono-valent (M⁺) cation and at least one di-valent (M⁺⁺) cation; or (b) 0.1 wt. % to 60 wt. % of a combination of (b1) water soluble salt of carboxymethyl cellulose (CMC) functionalized with at least one mono-valent (M⁺) cation and at least one di-valent (M⁺⁺) cation, and (b2) water soluble salt of carboxymethyl cellulose (CMC) functionalized with at least one mono-valent (M⁺) cation, (ii) 0.1 wt. % to 60 wt. % of a maleic acid or maleic anhydride co-polymer of C1 to C4 alkyl vinyl ether and maleic acid or anhydride monomers in substantially alternating monomer structure and having 50 to 90 wt. % of the acid moieties in the polymer converted to a metal salt(s) or mixed salts; and (iii) 0.1 wt. % to 80 wt. % of at least one additional ingredient.

In another aspect, the present application provides a toothpaste composition comprising: (i) (a) 0.1 wt. % to 20 wt. % of water soluble salt of carboxymethyl cellulose (CMC) functionalized with at least one mono-valent (M⁺) cation and at least one di-valent (M⁺⁺) cation; or (b) 0.1 wt. % to 20 wt. % of a combination of (b1) water soluble salt of carboxymethyl cellulose (CMC) functionalized with at least one mono-valent (M⁺) cation and at least one di-valent (M⁺⁺) cation, and (b2) water soluble salt of carboxymethyl cellulose (CMC) functionalized with at least one mono-valent (M⁺) cation, (ii) 0.1 wt. % to 60 wt. % of orally acceptable solvent or carrier selected from water, glycerin, polyethylene glycol, polypropylene glycol, sorbitol, PEG, ethylene oxide-propylene oxide polymers or combination thereof, and (iii) 0.1 wt. % to 20 wt. % of orally acceptable polymers of monomers including but not limited to maleic anhydride, maleic acid, C1 to C4 alkyl vinyl ethers, alkylene oxides, alkyl lactams, triglycerides, animal or vegetable oils, or combinations thereof, and (iv) 0.1-80 wt. % of an additional ingredient including but not limited to abrasives selected from the group comprising silica, calcium carbonate, calcium bicarbonate, sodium carbonate, sodium bicarbonate and combinations thereof, anti-microbial agents, anti-plaque agents, anti-tartar agents, anti-biofilm agents, anti-sensitivity agents, anti-gingivitis agents, actives selected from the group comprising fluoride providing compounds, dispersants, deposition agents, encapsulants, flavors, foam enhancers, film forming polymers, humectants, minerals, mouth feel agents, peroxides, pH modifiers, preservatives, rheology modifiers, salts, sweeteners, colorants, whitening agents, surfactants selected from the group comprising anionic, cationic, non-ionic or zwitterionic surfactants or combination thereof, strain removal agents, stain prevention agents, stabilizers, thickeners, vitamins and, zinc salts of organic and inorganic acids, or combinations thereof.

In another aspect, the present application provides a functionalized carboxymethyl cellulose obtained by ion-exchange process comprising a reaction of (i) at least one mono-valent (M⁺) carboxymethyl cellulose salt(s); and (ii) one or more di-valent (M⁺⁺) metal salt(s); wherein the di-valent (M⁺⁺) salt is selected from the group consisting of M⁺⁺ chloride or M⁺⁺ sulfate or M⁺⁺ carbonate, wherein the weight ratio of M⁺ to M⁺⁺ in the ion-exchanged product CMC is from about 1:75 to about 75:1.

In yet another aspect, the present application provides a method for preparing mono-valent-di-valent (M⁺-M⁺⁺)-carboxymethyl cellulose, the method comprising: (i) preparing an aqueous solution of (a) M⁺⁺ chloride, M⁺⁺ sulfate or M⁺⁺ carbonate and (b) C₂-C₄ alcohol; (ii) adding sodium (M⁺)-carboxymethyl cellulose to step (i) and stirring the resultant for at least 30 minutes at an ambient temperature; (iii) filtering the resultant of step (ii) to obtain wet cake; (iv) stirring the wet cake of step (iii) in a hydroalcoholic solution for at least 30 minutes; (v) repeating the step (iv) for at least 3 times to obtain pure wet cake of M⁺-M⁺⁺-carboxymethyl cellulose; (vi) drying the wet cake of step (v) on a fluid-bed dryer for at least 15-30 minutes at 50-70° C. to obtain dry M⁺-M⁺⁺-carboxymethyl cellulose; and (vii) grinding the resultant of step (vi) to isolate dry powder of M⁺-M⁺⁺-carboxymethyl cellulose.

### BRIEF DESCRIPTION OF THE DRAWINGS

In addition to the cited advantages and objects of the disclosure, one or more descriptions of the disclosure briefly summarized can be added by reference to certain embodiments thereof which are illustrated in the appended drawings. These drawings form part of the specification. However, it is to be noted that the appended drawings illustrate preferred embodiments of the disclosure and therefore are not limiting in their scope.

FIG. 1 illustrates adhesion performance of Sodium-Magnesium-CMC (Na⁺-Mg⁺⁺-CMC) containing denture adhesive formulae (DA29-31).

FIG. 2 illustrates total adhesion of Sodium-Magnesium-CMC (Na⁺-Mg⁺⁺-CMC) containing denture adhesive formulae (DA29-31).

FIG. 3 illustrates adhesion performance of Sodium-Calcium-CMC (Na⁺—Ca⁺⁺-CMC) containing denture adhesive formulae (DA35-38).

FIG. 4 illustrates total adhesion of Sodium-Calcium-CMC (Na⁺—Ca⁺⁺-CMC) containing denture adhesive formulae (DA35-38).

FIG. 5 illustrates adhesion performance of Sodium-Calcium-CMC (Na⁺—Ca⁺⁺-CMC) containing denture adhesive formulae (DA39-42).

FIG. 6 illustrates total adhesion of Sodium-Calcium-CMC (Na⁺—Ca⁺⁺-CMC) containing denture adhesive formulae (DA39-42).

FIG. 7 illustrates adhesion performance of Sodium-Calcium-CMC (Na⁺—Ca⁺⁺-CMC) containing denture adhesive formulae (DA43-46).

FIG. 8 illustrates total adhesion of Sodium-Calcium-CMC (Na⁺—Ca⁺⁺-CMC) containing denture adhesive formulae (DA43-46).

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “degree of substitution” or “D.S.” relates to the average number of hydroxyl groups substituted per anhydroglucose unit.

As used herein, the term “mono-valent cation” refers to a cation having a valency of one. Accordingly, the term “di-valent cation” refers to a cation having a valency of two. The mono-valent cation is selected from the group consisting of sodium, potassium, and lithium. Most preferably the mono-valent cation is sodium. The di-valent cation is selected from the group consisting of calcium, magnesium, zinc, tin, copper, and combinations thereof. More preferably, the di-valent cation is calcium, magnesium, zinc, and combinations thereof.

In one non-limiting embodiment, the present application provides a carboxylated polysaccharide functionalized with at least one mono-valent (M⁺) cation and at least one di-valent (M⁺⁺) cation. The polysaccharide is selected from the group comprising carboxymethyl cellulose (CMC), carboxymethyl inulin, carboxymethyl guar and carboxy methyl starch.

As used herein, the term “functionalized” with reference to “carboxylated polysaccharides” and in particular to carboxymethyl cellulose of this application refers to the presence of mono-valent and one or more di-valent cations on the polysaccharide or the carboxymethylcellulose. Various mono-valent and di-valent cations may be introduced in a carboxymethylcellulose by way of one or more functionalization reactions known to a person having ordinary skill in the art. Non-limiting examples of functionalization reactions include ion-exchange process comprising a reaction of (i) at least one mono-valent (M⁺) carboxymethyl cellulose salt(s); and (ii) one or more di-valent (M⁺⁺) metal salt(s); wherein the di-valent (M⁺⁺) salt is selected from the group consisting of M⁺⁺ chloride or M⁺⁺ sulfate or M⁺⁺ carbonate wherein the weight ratio of M⁺ to M⁺⁺ in the final ion exchanged carboxymethyl cellulose (CMC) polymer is from about 1:70 to about 70:1.

Accordingly, the functionalized carboxylated polysaccharides are used in various oral care compositions selected from mucoadhesive compositions, toothpaste/dentrifice compositions, oral therapeutic compositions, mouth rinses and denture adhesive compositions.

In another non-limiting embodiment, the present application provides a functionalized carboxylated polysaccharide, a water-soluble salt of carboxymethyl cellulose (CMC) functionalized with at least one mono-valent (M⁺) cation and at least one di-valent (M⁺⁺) cation.

The term “functionalized carboxymethyl cellulose” refers to low to medium or medium to high molecular weight carboxymethyl cellulose (CMC) functionalized with at least one mono-valent (M⁺) cation and at least one di-valent (M⁺⁺) cation, wherein, the mono-valent cation is selected from the group consisting of sodium, potassium, and lithium and di-valent cation is selected from the group consisting of calcium, magnesium, zinc, tin, copper, and combinations thereof.

The carboxymethyl cellulose functionalized with at least one mono-valent and at least one di-valent cation has a structure of (I):

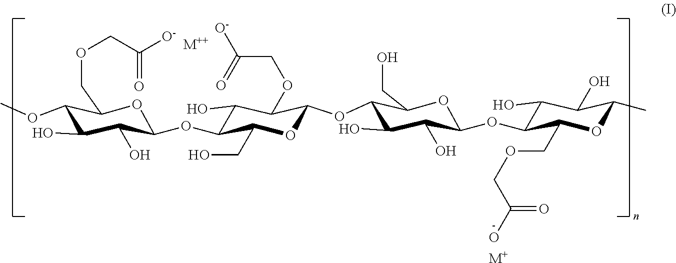

CMC functionalized with mono-valent cation and di-valent cation wherein,
M⁺ isa mono-valent cation selected from the group consisting of sodium, potassium, and lithium; and
M⁺⁺ is a di-valent cation selected from the group consisting of calcium, magnesium, zinc, tin, copper, and combinations thereof; and
wherein n≥2 to n≤2000, M⁺=0.1 to 10 wt. % and M⁺⁺=0.1 to 7.5 wt. % of the carboxymethyl cellulose.

In another non-limiting embodiment, the present application discloses a functionalized carboxymethyl cellulose containing 0.1 to 10% by weight of mono-valent cation and 0.1 to 10% by weight of one or more di-valent cations. In another embodiment, the di-valent cation is present in an amount of from about 0.2 to 7.5 wt. % of the total CMC content.

In another non-limiting embodiment, the present application discloses mono-valent cation (M⁺) to di-valent cation (M⁺⁺) weight ratio of from about 1:75 to about 75:1 in the final functionalized CMC polymer. In some embodiments, the weight ratio of M⁺ to M⁺⁺ is from about 1:3 to about 3:1; or from about 1:5 to about 5:1; or from about 1:10 to about 10:1; or from about 1:20 to about 20:1; or from about 1:30 to about 30:1; or from about 1:40 to about 40:1; or from about 1:50 to about 50:1; or from about 1:60 to about 60:1; or from about 1:70 to about 70:1 in the final functionalized CMC polymer. In another non-limiting embodiment, the preferred weight ratio of M⁺ to M⁺⁺ is from about 1:3 to about 3:1; or from about 1:5 to about 5:1; or from about 1:10 to about 10:1. In a preferred embodiment, the mono-valent and di-valent cations are present in the weight ratio of 75 to 1:1 to 75 in the final functionalized CMC polymer.

In another non-limiting embodiment, the present application provides a modified carboxymethyl cellulose (CMC), wherein the CMC is optionally further combined with carboxymethyl cellulose (CMC) functionalized with at least one mono-valent (M⁺) cation. Accordingly, the functionalized carboxymethyl cellulose having at least one mono-valent cation has a structure of (II):

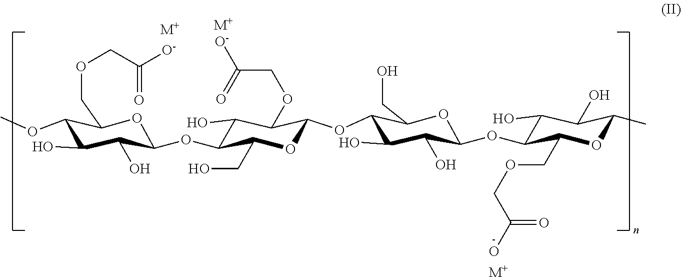

CMC functionalized with mono-valent cation
wherein,
M⁺ isa mono-valent cation selected from the group consisting of sodium, potassium, and lithium; and wherein n≥2 to n≤2000, M⁺=0.1 to 10 wt. % of the carboxymethyl cellulose.

In another non-limiting embodiment, the present application discloses biodegradable functionalized carboxymethyl cellulose (CMC) comprising at least one mono-valent (M⁺) cation and at least di-valent (M⁺⁺) cation. The mono-valent (M⁺) cation is selected from the group consisting of sodium, potassium, and lithium. The di-valent (M⁺⁺) cation is selected from the group consisting of calcium, magnesium, zinc, copper, tin, and combinations thereof. Further, the functionalized carboxymethyl cellulose (CMC) can be in dry powder form.

In another non-limiting embodiment, the functionalized carboxymethyl cellulose has a degree of substitution in the range from about 0.4 to about 0.5; or from about 0.5 to about 0.6; or from about 0.6 to about 0.7; or from about 0.7 to about 0.8; or from about 0.8 to about 0.9, or from about 0.9 to about 1.0, or from about 1.0 to about 1.1, or from about 1.1 to about 1.2. In a preferred embodiment, the degree of substitution is in the range from about 0.4 to about 0.7. In another preferred embodiment, the modified cellulose has DS of about 0.4 to about 1.2.

In another non-limiting embodiment, the present functionalized carboxymethyl cellulose has low to medium or medium to high molecular weight ranges. Low to medium molecular weight range refers to functionalized carboxymethyl cellulose (CMC) having about 2,000 to about 200,000 Daltons weight average molecular weight. Similarly, medium to high molecular weight range refers to functionalized carboxymethyl cellulose (CMC) having about 200,000 to about 2,000,000 Daltons weight average molecular weight.

In some embodiments, the average molecular weight is in the range of from about 2000 to about 2,000,000 Daltons. In some embodiments, the molecular weight ranges from about 10000 to about 15000 Daltons; or from about 15000 to about 20000 Daltons; or from about 20000 to about 300000; or from about 30000 to about 40000 Daltons; or from about 40000 to about 50000 Daltons; or from about 50000 to about 60000 Daltons; or from about 60000 to about 70000 Daltons; or from about 70000 to about 80000 Daltons. In some embodiments, the molecular weight ranges from about 80000 to about 90000 Daltons; or from about 90000 to about 100000 Daltons; or from about 100000 to about 200000 Daltons; or from about 200000 to about 300000 Daltons; or from about 300000 to about 400,000 Daltons, or from about 400,000 to 500,000 Daltons, or from about 500,000 to 600,000 Daltons, or from about 600,000 to 700,000 Daltons, or from about 700,000 to 800,000 Daltons, or from about 800,000 to 900,000 Daltons, or from about 900,000 to 1,000,000 Daltons, or from about 1,000,000 to 1,100,000 Daltons. In a preferred embodiment, the functionalized carboxymethyl cellulose has a weight average molecular weight in the range of 2000 to 1,500,000 Daltons. In another preferred embodiment, the functionalized carboxymethyl cellulose has a weight average molecular weight in the range of 80,000 to 1,300,000 Daltons.

Accordingly, the functionalized carboxymethyl cellulose (CMC) is biodegradable.

In another non-limiting embodiment, the functionalized water soluble carboxymethyl cellulose is selected from the group comprising: (i) sodium-calcium carboxymethyl cellulose (Na⁺—Ca⁺⁺-CMC), (ii) sodium-magnesium carboxymethyl cellulose (Na⁺-Mg⁺⁺-CMC), (iii) sodium-zinc carboxymethyl cellulose (Na⁺—Zn⁺⁺-CMC), (iv) sodium-calcium-zinc carboxymethyl cellulose (Na⁺—Ca⁺⁺—Zn⁺⁺-CMC), (v) sodium-calcium-magnesium carboxymethyl cellulose (Na⁺—Ca⁺⁺—Mg⁺⁺-CMC), (vi) sodium-magnesium-zinc carboxymethyl cellulose (Na⁺-Mg⁺⁺—Zn⁺⁺-CMC), (vii) sodium-tin-carboxymethyl cellulose (Na⁺—Sn⁺⁺-CMC, (viii) sodium-calcium-tin-carboxymethyl cellulose (Na⁺—Ca⁺⁺—Sn⁺⁺-CMC), (ix) sodium-magnesium-tin-carboxymethyl cellulose (Na⁺—Mg⁺⁺—Sn⁺⁺-CMC), or (x) sodium-zinc-tin-carboxymethyl cellulose (Na⁺—Zn⁺⁺—Sn⁺⁺-CMC). Further, the sodium carboxymethyl cellulose (Na⁺-CMC) can be combined with at least one functionalized carboxymethyl cellulose (CMC) selected from combination of CMC monovalent and divalent salt combinations described herein above.

In another non-limiting embodiment, the functionalized water soluble carboxymethyl cellulose is selected from the group comprising: (i) potassium-calcium-carboxymethyl cellulose (K⁺—Ca⁺⁺-CMC), (ii) potassium-magnesium-carboxymethyl cellulose (K⁺-Mg⁺⁺-CMC), (iii) potassium-zinc-carboxymethyl cellulose (K⁺—Zn⁺⁺-CMC), (iv) potassium-tin-carboxymethyl cellulose (K⁺—Sn⁺⁺-CMC), (v) potassium-calcium-zinc-carboxymethyl cellulose (K⁺—Ca⁺⁺—Zn⁺⁺-CMC), (vi) potassium-calcium-magnesium-carboxymethyl cellulose (K⁺-Ca⁺⁺-Mg⁺⁺-CMC), (vii) potassium-calcium-tin-carboxymethylcellulose (K⁺—Ca⁺⁺—Sn⁺⁺-CMC), (viii) potassium-magnesium-zinc-carboxymethylcellulose (K⁺-Mg⁺⁺—Zn⁺⁺-CMC), (ix) potassium-magnesium-tin-carboxy methyl cellulose (K⁺-Mg⁺⁺—Sn⁺⁺-CMC), and (x) potassium-zinc-tin-carboxymethyl cellulose (K⁺—Zn⁺⁺—Sn⁺⁺-CMC).

In another non-limiting embodiment, the functionalized water soluble carboxymethyl cellulose is selected from the group comprising: (i) lithium-calcium-carboxymethyl cellulose (Li⁺—Ca⁺⁺-CMC), (ii) lithium-magnesium-carboxymethyl cellulose (Li⁺-Mg⁺⁺-CMC), (iii) lithium-zinc carboxymethyl cellulose (Li⁺—Zn⁺⁺-CMC), (iv) lithium-tin carboxymethyl cellulose (Li⁺—Sn⁺⁺-CMC), (v) lithium-calcium-zinc-carboxymethyl cellulose (Li⁺—Ca⁺⁺—Zn⁺⁺-CMC), (vi) lithium-calcium-magnesium-carboxymethyl cellulose (Li⁺-Ca⁺⁺-Mg⁺⁺-CMC), (vii) lithium-calcium-tin-carboxymethyl cellulose (Li⁺—Ca⁺⁺—Sn⁺⁺-CMC), (viii) lithium-magnesium-zinc-carboxymethyl cellulose (Li⁺-Mg⁺⁺—Zn⁺⁺-CMC), (ix) lithium-magnesium-tin-carboxymethyl cellulose (Li⁺-Mg⁺⁺—Sn⁺⁺-CMC), and (x) lithium-zinc-tin-carboxymethyl cellulose (Li⁺—Zn⁺⁺—Sn⁺⁺-CMC).

In another non-limiting embodiment, the functionalized CMC is sodium-calcium-carboxymethyl cellulose (Na⁺—Ca⁺⁺-CMC) or sodium-magnesium-carboxymethyl cellulose (Na⁺—Mg⁺⁺-CMC) or sodium-zinc-carboxymethyl cellulose (Na⁺—Zn⁺⁺-CMC).

In another non-limiting embodiment, the functionalized carboxymethyl cellulose (CMC) is a combination of (i) sodium carboxymethyl cellulose (Na⁺-CMC) with (ii) (a) sodium-calcium carboxymethyl cellulose (Na⁺—Ca⁺⁺-CMC) or (b) sodium-magnesium-carboxymethyl cellulose (Na⁺-Mg⁺⁺-CMC) or (c) sodium-zinc-carboxymethyl cellulose (Na⁺—Zn⁺⁺-CMC).

The functionalized carboxymethyl cellulose is in a microcrystalline, a granular, a powder or in a solution state.

In another non-limiting embodiment, the functionalized carboxymethyl cellulose (CMC) is used in oral care composition, pharmaceutical composition, home care composition, industrial composition, skin care composition, food and nutraceutical composition, coating composition, and oil & energy composition.

The functionalized carboxymethyl cellulose (CMC) is used in oral care composition selected from mucoadhesive composition, toothpaste/dentrifice composition, oral therapeutic composition, mouth rinse composition and denture adhesive composition.

In another non-limiting embodiment, the present application provides a mucoadhesive composition comprising: (a) 0.1 wt. % to 60 wt. % of water soluble salt of carboxymethyl cellulose (CMC) functionalized with at least one mono-valent (M⁺) cation and at least one di-valent (M⁺⁺) cation; or (b) 0.1 wt. % to 60 wt. % of a combination of (b1) water soluble salt of carboxymethyl cellulose (CMC) functionalized with one mono-valent (M⁺) cation and at least one di-valent (M⁺⁺) cation, and (b2) water soluble salt of carboxymethyl cellulose (CMC) functionalized with at least one mono-valent (M⁺) cation, (ii) 0.1 wt. % to 60 wt. % of a maleic acid or maleic anhydride co-polymer of C₁ to C₄ alkyl vinyl ether and maleic acid or anhydride monomers in substantially alternating monomer structure and having 50 to 90 wt. % of the acid moieties in the polymer converted to a metal salt(s) or mixed salts; and (iii) 0.1 wt. % to 80 wt. % of at least one additional ingredient.

In another embodiment, the present application discloses a mucoadhesive composition comprising functionalized carboxymethyl cellulose present in an amount of from about 0.1 wt. % to about 1 wt. %; or from about 1 wt. % to about 2.5 wt. %; or from about 2.5 wt. % to about 5 wt. %; or from about 5 wt. % to about 10 wt. %; or from about 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. % or from about 80 wt. % to about 85 wt. % or 85 wt. % to about 90 wt. %; or 90 wt. % to about 95 wt. %; or 95 wt. % to about 99.9 wt. % based on the total weight of the composition.

The mucoadhesive composition comprises water soluble functionalized carboxymethyl cellulose (CMC) having a degree of substitution in the range of 0.4 to 1.2. The water soluble functionalized biodegradable carboxymethyl cellulose (CMC) has a degree of substitution in the range of 0.4 to 0.7.

The functionalized carboxymethyl cellulose has a weight average molecular weight in the range of 2000 to 2000,000 Daltons.

In another non-limiting embodiment, the mucoadhesive composition comprises maleic acid or maleic anhydride co-polymer of C1 to C4 alkyl vinyl ether and maleic acid or anhydride monomers in substantially alternating monomer structure and having 50 to 90 wt. % of the acid moieties in the polymer converted to a salt or mixed salts of at least one mono-valent cation (M⁺) and at least one di-valent cation (M⁺⁺). Accordingly, the maleic acid or maleic anhydride copolymer comprises maleic acid or maleic anhydride and a C1-C4 alkyl vinyl ether, having a predetermined weight average molecular weight of about 500,000 to 3,000,000 made by copolymerizing about 50 mole percent of maleic acid or maleic anhydride, about 50 mole percent of a C1-C4 alkyl vinyl ether, in the presence of a free radical initiator, at about 50° to 150° C. in a solvent or solvent free process to produce a uniform, fine powder having substantially no residual maleic anhydride.

The maleic acid or maleic anhydride and a C1-C4 alkyl vinyl ether is a methyl vinyl ether-maleic acid copolymer or methyl vinyl ether-maleic anhydride copolymer has a number average molecular weight of between about 50,000 and about 500,000 wherein about 50 to about 100 wt. % of the carboxyl units in the polymer are converted to a mixture of metal salts selected from the group consisting of calcium, sodium, strontium, zinc, magnesium, iron, boron, aluminum, potassium, vanadium, chromium, manganese, nickel, copper, yttrium, titanium, and mixtures thereof. Preferably, the salts of the copolymer of methyl vinyl ether-maleic acid or methyl vinyl ether-maleic anhydride has a specific viscosity of from about 2.5 to 5.0 when measured as a 1% w/v solution in methyl ethyl ketone (MEK) solution at 25° C. The copolymer of methyl vinyl ether-maleic acid or methyl vinyl ether-maleic anhydride or salts thereof comprises from about 10 to about 66 wt. % of said composition. Preferably, the methyl vinyl ether-maleic acid or methyl vinyl ether-anhydride copolymer has a weight average molecular weight of from about 700,000 to about 3,000,000.

In another embodiment, the present mucoadhesive composition provides an additional ingredient is selected from the group consisting of abrasives, anti-caking agents, anti-fungal agents, anti-microbial agents, anesthetic agents, antioxidants, anti-biotics, anti-inflammatory agents, binder, buffers, colors, cooling agents, dentinal desensitizing agents, dispersants, enzymes, foaming agents, flavors, fillers, fragrances, gelling agent, humectants, hydrophobic carriers, hydrophilic non-oil components, preservatives, pigment, plasticizers, pain relieving agents, sweeteners, thickening agents, viscosity modifiers, vehicles, surfactants, stabilizers, sensates and mixtures thereof. Preferably, the additional ingredient is selected from petrolatum, mineral oil, olive oil, vegetable oil, silicone, glycerin, polyethylene glycol, propylene glycol, polypropylene glycol, poly(ethylene oxide-propylene oxide) copolymer, diethylene glycol, triethylene glycol, sorbitol, water, and mixtures thereof.

In another non-limiting embodiment, the mucoadhesive composition is a buccal composition, an oral composition, a vaginal composition, a nasal composition, a rectal composition, an ocular composition, a sublingual composition, a palatal composition, and a denture adhesive composition. The mucoadhesive composition is formulated into an ointment, a lotion, a liquid, a solid, a semisolid, an emulsion, a powder, a paste, a dispersion, a gel, a patch, an aerosol, or a spray.

In another non-limiting embodiment, the present application provides a denture adhesive composition comprising: (i) (a) 0.1 wt. % to 60 wt. % of water soluble salt of carboxymethyl cellulose (CMC) functionalized with at least one mono-valent (M⁺) cation and at least one di-valent (M⁺⁺) cation; or (b) 0.1 wt. % to 60 wt. % of a combination of (b1) water soluble salt of carboxymethyl cellulose (CMC) functionalized with one mono-valent (M⁺) cation and at least one di-valent (M⁺⁺) cation, and (b2) water soluble salt of carboxymethyl cellulose (CMC) functionalized with at least one mono-valent (M⁺) cation, (ii) 0.1 wt. % to 60 wt. % of a maleic acid or maleic anhydride co-polymer of C₁ to C₄ alkyl vinyl ether and maleic acid or anhydride monomers in substantially alternating monomer structure and having 50 to 90 wt. % of the acid moieties in the polymer converted to a metal salt(s) or mixed salts; and (iii) 0.1 wt. % to 80 wt. % of at least one additional ingredient. The denture adhesive composition comprises water soluble functionalized carboxymethyl cellulose (CMC) has a degree of substitution in the range of 0.4 to 0.7. Preferably, the denture adhesive composition comprises water soluble functionalized carboxymethyl cellulose (CMC) has a degree of substitution in the range of 0.4 to 1.2.

The functionalized carboxymethyl cellulose has a weight average molecular weight in the range of 2000 to 2000,000 Daltons. The maleic acid or maleic anhydride co-polymer of C₁ to C₄ alkyl vinyl ether and maleic acid or anhydride monomers is mixed sodium and calcium salt of methyl vinyl ether and maleic anhydride having a specific viscosity of from about 2.5 to 5.0 when measured as a 1% w/v solution in methyl ethyl ketone (MEK) solution at 25° C.

In another non-limiting embodiment, the present application provides a toothpaste composition comprising: (i) (a) 0.1 wt. % to 20 wt. % of water soluble salt of carboxymethyl cellulose (CMC) functionalized with at least one mono-valent (M⁺) cation and at least one di-valent (M⁺⁺) cation; or (b) 0.1 wt. % to 20 wt. % of a combination of (b1) water soluble salt of carboxymethyl cellulose (CMC) functionalized with at least one mono-valent (M⁺) cation and at least one di-valent (M⁺⁺) cation, and (b2) water soluble salt of carboxymethyl cellulose (CMC) functionalized with at least one mono-valent (M⁺) cation, (ii) 0.1 wt. % to 60 wt. % of orally acceptable solvent or carrier including but not limited to water, glycerin, polyethylene glycol, polypropylene glycol, sorbitol, or combination thereof, 0.1 wt. % to 20 wt. % of orally acceptable polymers of monomers including but not limited to maleic anhydride, maleic acid, C₁ to C₄ alkyl vinyl ethers, alkylene oxides, alkyl lactams, triglycerides, animal or vegetable oils, or combinations thereof, and (iii) 0.1-80 wt. % of an additional ingredient including but not limited to abrasives selected from the group comprising calcium carbonate, calcium bicarbonate, silica, sodium carbonate, sodium bicarbonate and combinations thereof, surfactants selected from the group comprising anionic, cationic, non-ionic, zwitterionic surfactants and combination thereof, actives selected from fluoride providing compounds, zinc salts of organic and inorganic acids, flavors, foam enhancers, stabilizers, film forming polymers, peroxides, rheology modifiers, thickeners, salts, sweeteners, colorants, whitening agents, anti-microbial agents, anti-plaque agents, anti-tartar agents, anti-biofilm agents, anti-sensitivity agents, anti-gingivitis agents, mouth feel agents, encapsulants and compounds, strain removal agents, stain prevention agents, vitamins and minerals humectants, preservatives, dispersants, deposition agents, pH modifiers, and combinations thereof.

One or more oral care active ingredients in the oral care composition of the present disclosure can be selected from the group consisting of an analgesic, an antibacterial, an anti-calculus agents, an antibiotic, an antioxidant, odor or breath freshening agents, a cooling agent, a caffeine, a drug, a desensitizing agent, a dental remineralization agent, an enzyme, herbal agents, medicaments, a mineral, a peptide, pharmaceutical agent, a probiotic, a preservative, a sensate, taste masking agents, spices, a therapeutic agent, throat-soothing agent, vitamins, warming agents, and mixtures thereof.

Non-limiting examples of humectants may be or include glycerin, propylene glycol, and combinations thereof. The tooth paste composition may include one or more whitening agents. not limiting to, hydrogen peroxide or one or more sources of hydrogen peroxide.

Non-limiting examples of antibacterial agents can include zinc ion sources such as zinc acetate, zinc citrate, zinc gluconate, zinc glycinate, zinc oxide, zinc sulfate, and sodium zinc citrate; phthalic acid and salts thereof such as magnesium monopotassium phthalate; hexetidine; octenidine; sanguinarine; benzalkonium chloride; domiphen bromide; alkylpyridinium chlorides such as cetylpyridinium chloride (CPC) (including combinations of CPC with zinc and/or enzymes), tetra decyl pyridinium chloride and N-tetradecyl-4-ethylpyridinium chloride; iodine; sulfonamides, bisbiguanides such as alexidine, chlorhexidine; digluconate; piperidino derivatives such as delmopinol and octapinol, magnolia extract, grapeseed extract, raspberry ketone, menthol, geraniol, citral, eucalyptol, eugenol, stannous fluoride, sodium mono magnolia bark extracts, Chinese traditional medicines, thymol, 4-isopropyl m-cresol (IPMP, o-cymen-5-ol) antibiotics such as amoxicillin, tetracycline, doxycycline, minocycline, metronidazole, neomycin, kanamycin and clindamycin, and the like. A further illustrative list of useful antibacterial agents is provided in U.S. Pat. No. 5,776,435 to Gaffar et al.

Non-limiting examples of anti-microbial agents include, halogenated diphenyl ether, 2,4,4-trichloro-2′-hydroxy-diphenyl ether, 2,2-dihydroxy-5,5-dibromo-diphenyl ether, 2,2-methylenebis-4 (4-chloro-6-bromo-phenol), halogenated salicylanilides and halogenated cabanilides, stannous chloride, zinc lactate, zinc citrate, zinc oxide.

Non-limiting examples of flavorants include those flavors known to the skilled artisan, such as natural and artificial flavors. These flavorings chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also, useful flavorings are artificial, natural, and synthetic fruit flavors such as vanilla, and citrus oils including lemon, orange, lime, grapefruit, and fruit essences including apple, pear, peach, grape, blueberry, strawberry, raspberry, cherry, plum, pineapple, apricot and so forth. These flavoring agents can be used in liquid or solid form and can be used individually or in admixture. Commonly used flavors include mints such as peppermint, menthol, spearmint, artificial vanilla, cinnamon derivatives, and various fruit flavors, whether employed individually or in admixture.

Flavors can also provide breath freshening properties, particularly the mint flavors when used in combination with the cooling agents, described herein below. Other useful flavorings include aldehydes, esters and ketones such as cinnamyl acetate, cinnamaldehyde, citral diethyl-acetal, dihydrocarvyl acetate, eugenyl formate, raspberry ketone p-methyl-amisol, and so forth may be used. Generally, any flavoring or food additive such as those described in Chemicals Used in Food Processing, publication 1274, pages 63-258, by the National Academy of Sciences, may be used. This publication is incorporated herein by reference. This can include natural as well as synthetic flavors.

Non-limiting examples of suitable animal and vegetable oils can include, but are not limited, sunflower oil, corn oil, soy oil, avocado oil, jojoba oil, squash oil, raisin seed oil, sesame seed oil, walnut oil, fish oil, glycerol tricaprocaprylate, purcellin oil, liquid jojoba, and blends thereof. Also suitable are natural oils such as oils of eucalyptus, lavender, vetiver, litsea cubeba, lemon, sandalwood, rosemary, chamomile, savory, nutmeg, cinnamon, hyssop, caraway, orange, geranium, cade, bergamot, and blends thereof. There are five commonly available vegetable oils which contain greater than 50% linoleic acid groups (as their glycerol esters). These are, in decreasing order of linoleic content: Safflower Oil (75%); Sunflower Oil (67.8%); Corn Oil (56%); Cottonseed Oil (55%) and Soybean Oil (52%).

Non-limiting examples of suitable vitamins or minerals can include vitamin A, vitamin C, vitamin D, vitamin E, vitamin K, vitamin B6, vitamin B12, thiamine, riboflavin, biotin, folic acid, niacin, pantothenic acid, sodium, potassium, calcium, magnesium, iron, copper, zinc, selenium, manganese, choline, chromium, molybdenum, cobalt, and combinations thereof, can be used.

Similarly, non-limiting examples of whitening agents that can be used in the present oral care composition include, but are not limited to, dyes, polyphosphates, phytic acid and its' salts, complexed bleaching, or bleaching agents. Suitable bleaching or whitening agents include peracids, peroxide compounds. Peroxides are believed to whiten the teeth by releasing hydroxyl radicals capable of breaking down the plaque-stain complex into a form that can be flushed away or removed by abrasives.

Further, a film-forming polymer can be included in the present oral care composition. Suitable examples of such film forming polymers can include, but are not limited, to synthetic anionic polymeric polycarboxylate (SAPP), such a PVM/MA copolymer (Gantrez S-97, Ashland Inc.). Such polymers are also described in the U.S. Pat. Nos. 5,334,375 and 5,505,933. SAPP's have previously been described as useful for dentin sensitivity reduction. Moreover, SAPP's have previously been described as antibacterial-enhancing agents, which enhance delivery of an antibacterial agent to oral surfaces, and which enhance the retention of the antibacterial agent on oral surfaces. It is well within the contemplation of the present invention that film-forming polymers, such as PVM/MA copolymer, can be employed in the compositions of the present invention as a means of reducing stain formation.

Non-limiting examples of the sweetening agents can include, but are not limited to, sucrose, glucose, saccharin, dextrose, levulose, lactose, mannitol, sorbitol, fructose, maltose, xylitol, erythritol, saccharin salts, thaumatin, aspartame, D-tryptophan, dihydrochalcones, acesulfame, sucralose and cyclamate salts, especially sodium cyclamate and sodium saccharin, and mixtures thereof.

The oral care composition according to the present disclosure can further comprise abrasives. Suitable and non-limiting examples of such abrasives can include silica abrasives, such as standard cleaning silicas, high cleaning silicas or any other suitable abrasive silicas. Additional examples of abrasives that can be used in addition to or in place of the silica abrasives include, for example, a calcium phosphate abrasive, e.g., tricalcium phosphate (Ca₃(PO₄)₂), hydroxyapatite (Ca₁₀(PO₄)₆ (OH)₂), or dicalcium phosphate dihydrate CaHPO₄·2H₂O or calcium pyrophosphate; calcium carbonate abrasive; or abrasives such as sodium metaphosphate, potassium metaphosphate, aluminum silicate, calcined alumina, bentonite or other siliceous materials, or combinations thereof. The silica component of the present silica substrate is an amorphous precipitated silica. Precipitated silicas include the following products available from the J. M. Huber Corporation, Edison, N. J.: Zeodent® 103, Zeodent® 113, Zeodent® 114, Zeodent® 115, Zeodent® 118, Zeodent® 119, Zeodent® 165, and Zeodent® 9175.

The oral care composition according to the present disclosure can further comprise suitable anionic surfactants. Suitable examples of such surfactants can include, but are not limited to, water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms, such as sodium lauryl sulfate and sodium coconut monoglyceride sulfonate. Other suitable anionic surfactants are sarcosinates, such as sodium lauroyl sarcosinate, taurates, sodium lauryl sulfoacetate, sodium lauroyl isethionate, sodium lauryl carboxylate, and sodium dodecyl benzenesulfonate, and mixtures thereof. Other useful anionic surfactant includes sarcosinate surfactants, isethionate surfactants and taurate surfactants. Surfactants are alkali metal or ammonium salts of these surfactants, sodium and potassium salts of lauroyl sarcosinate, myristoyl sarcosinate, palmitoyl sarcosinate, stearyl sarcosinate, Amisoft CS-11 (amino acid and L-Glutamic Acid) and oleoyl sarcosinate.

Examples of suitable cationic surfactants are derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutyl phenoxyethyl-dimethyl benzyl ammonium chloride; coconut alkyltrimethyl ammonium nitrite; cetyl pyridinium fluoride etc.

Examples of suitable nonionic surfactants can include, but are not limited to, Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Examples of suitable zwitterionic surfactants can include, but not limited to derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

In another embodiment of the present disclosure, the oral care composition can further comprise rheology modifiers. Suitable examples of such thickening agents include, but not limiting to, water-soluble cellulose ethers such as sodium carboxymethylcellulose, sodium carboxymethyl hydroxyethyl cellulose and, hydroxyethyl cellulose, vinylpyrrolidone polymers (also referred to as N-vinylpyrrolidone, N-vinyl-2-pyrrolidone and N-vinyl-2-pyrrolidinone, polyvinyl pyrrolidone) as a monomeric unit. Other suitable thickening agents include carboxy vinyl polymers (carbomers), carrageenan, laponite and other natural gums such as gum karaya, xanthan gum, guar gum, gum arabic, and gum tragacanth. Colloidal magnesium aluminum silicate or finely divided silica can be used as part of the thickening agent to further improve texture. A class of other thickening or gelling agents includes a class of homopolymers of acrylic acid cross-linked with an alkyl ether of pentaerythritol or an alkyl ether of sucrose. In various preferred embodiments, the carrier may comprise polymers and/or copolymers of polyethylene glycol, of ethylene oxide/propylene oxide, and of silicone. If such copolymers/polymers are used, they may be selected from commercially available materials. Block copolymers of ethylene oxide propylene oxide are useful, but higher molecular weight, e.g., >5000 Da are preferred, e.g. including PLURA CARE (R) L1220 (available from BASF, Wyandotte, Mich., United States of America). Low or medium molecular weight polyethylene glycol, e.g., PEG 400, PEG 600, PEG 800, PEG 1000 and mixtures thereof are also useful. It is preferred that the carrier(s) provide a dentifrice with a viscosity of about 10,000 CPS to about 700,000 CPS, preferably about 30,000 CPS to about 300,000 CPS.

Non-limiting examples of “fluoride-providing compound” can include sodium fluoride, potassium fluoride, amine fluoride, ammonium fluoride, lead fluoride, manganese fluoride, a copper fluoride such as cuprous fluoride, zinc fluoride, barium fluoride, sodium fluorosilicate, ammonium fluorosilicate, sodium fluorozirconate, sodium monofluorophosphate, potassium fluoro zirconates, sodium monofluorophosphate, aluminum mono-fluorophosphate and aluminium di-fluorophosphate, and fluorinated sodium calcium pyrophosphate, a tin fluoride such as stannous fluoride, stannic fluoride or stannous chloro fluoride, and sodium hexafluoro stannate.

Accordingly, the toothpaste composition is an aqueous composition or anhydrous composition.

In another non-limiting embodiment, the present application discloses a process for preparing a functionalized carboxymethyl cellulose, the process includes ion-exchange process comprising a reaction of (i) at least one mono-valent (M⁺) carboxymethyl cellulose salt(s); and (ii) one or more di-valent (M⁺⁺) metal salt(s); wherein the di-valent (M⁺⁺) salt is selected from the consisting group of M⁺⁺ chloride or M⁺⁺ sulfate or M⁺⁺ carbonate, wherein the weight ratio of M⁺ to M⁺⁺ is from about 1:75 to about 75:1 in the final functionalized CMC polymer.

In another non-limiting embodiment, the present application provides a method for preparing mono-valent-di-valent (M⁺-M⁺⁺)-carboxymethyl cellulose, the method comprising: (i) preparing an aqueous solution of (a) M⁺⁺ chloride, M⁺⁺ sulfate or M⁺⁺ carbonate and (b) C2-C₄ alcohol; (ii) adding sodium (M⁺)-carboxymethyl cellulose to step (i) and stirring the resultant for at least 30 minutes at an ambient temperature; (iii) filtering the resultant of step (ii) to obtain wet cake; (iv) stirring the wet cake of step (iii) in a hydroalcoholic solution for at least 30 minutes; (v) repeating the step (iv) for at least 3 times to obtain pure wet cake of M⁺-M⁺⁺-carboxymethyl cellulose; (vi) drying the wet cake of step (v) on a fluid-bed dryer for at least 15-30 minutes at 50-70° C. to obtain dry M⁺-M⁺⁺-carboxymethyl cellulose; and (vii) grinding the resultant of step (vi) to isolate dry powder of M⁺-M⁺⁺-carboxymethyl cellulose.

The M⁺-M⁺⁺-carboxymethyl cellulose is sodium-calcium carboxymethyl cellulose. The M⁺⁺ chloride, M⁺⁺ sulfate or M⁺⁺ carbonate is calcium chloride, calcium sulfate or calcium carbonate. The C1-C4 alcohol is selected from methanol, ethanol, propanol, isopropanol, and butanol. The grinding is carried out in a mill using 0.75, 0.20, and 0.08 mm screens. The dry powder has a mean particle diameter in the range of 20 to 300 microns.

Therefore, the present functionalized carboxymethyl cellulose has multiple unique features namely: (i) has multi-valent cations with at least one di-valent (M⁺⁺) cation (such as calcium, magnesium, zinc and tin); (ii) has low to medium (2000 to 200,000 Daltons) or medium to high molecular weight (200,000 to 2,000,000 Daltons), (iii) is highly soluble in water; (iv) is biodegradable making it suitable for preparing mucoadhesive compositions including application in denture adhesives and tooth paste formulations.

Further, certain aspects of the present application are illustrated in detail by way of the following examples. Therefore, every example provided herein, must be construed as merely illustrative and not a limitation on the scope of the present invention in any way.

### Examples

Manufacturing and application of functionalized carboxymethyl cellulose directed to compositions containing a high molecular weight carboxymethyl cellulose functionalized with at least one mono-valent (M⁺) cation and at least one di-valent (M⁺⁺) cation, as well as to the process and method of producing mucoadhesive compositions are represented in the following examples.

### Example 1: Synthesis of Prototype Na⁺—Ca⁺⁺-CMC Polymer, XA-1517-050-1

4.35 grams of anhydrous calcium chloride was dissolved in 320 grams deionized water with stirring for 30 minutes. 1,280 grams of methanol was added with continual stirring. 128 grams of Aqualon™ CMC 7H3SXF grade carboxymethylcellulose supplied by Ashland Specialty Ingredients was added to this solution with continual stirring, creating a heterogenous slurry. This mixture was stirred for 20 hours before being filtered to obtain a wet cake. The wet cake was dispersed in 1,600 grams of 80/20 w/w methanol/water and stirred for 30 minutes before filtering again. This step was repeated two additional times and then a final wash in 1,600 grams of neat methanol was carried out. The final material was dried at 65° C. using a fluid bed dryer until total moisture content of the final product was under 5 weight percent.

### Example 2: Synthesis of Prototype Nat-Ca⁺⁺-CMC Polymer, XA-1517-050-2

13.40 grams of anhydrous calcium chloride was dissolved in 320 grams deionized water with stirring for 30 minutes. 1,280 grams of methanol was added with continual stirring. 128 grams of Aqualon™ CMC 7H3SXF grade carboxymethylcellulose supplied by Ashland Specialty Ingredients was added to this solution with continual stirring, creating a heterogenous slurry. This mixture was stirred for 20 hours before being filtered to obtain a wet cake. The wet cake was dispersed in 1,600 grams of 80/20 w/w methanol/water and stirred for 30 minutes before filtering again. This step was repeated two additional times and then a final wash in 1,600 grams of neat methanol was carried out. The final material was dried at 65° C. using a fluid bed dryer until total moisture content of the final product was under 5 weight percent.

### Example 3: Synthesis of Prototype Nat-Ca⁺⁺-CMC Polymer, XA-1517-078

120.00 grams of anhydrous calcium chloride was dissolved in 800 grams deionized water with stirring for 30 minutes. 800 grams of isopropanol was added with continual stirring. 180 grams of Aqualon™ CMC 7H3SXF grade carboxymethylcellulose supplied by Ashland Specialty Ingredients was added to this solution with continual stirring, creating a heterogenous slurry. This mixture was stirred for 20 hours before being filtered to obtain a wet cake. The wet cake was dispersed in 1,600 grams of 80/20 w/w methanol/water and stirred for 30 minutes before filtering again. This step was repeated two additional times and then a final wash in 1,600 grams of neat methanol was carried out. The final material was dried at 65° C. using a fluid bed dryer until total moisture content of the final product was under 5 weight percent.

TABLE 1
Characteristic properties of Na⁺—Ca⁺⁺-CMC polymer prototypes

| | | | | | | Viscosity | | pH |
|---|---|---|---|---|---|---|---|---|
| | | | | | Mol. | (1% in | Degree of | (@1% |
| Polymer | M⁺ | | M⁺⁺ | | Wt.(Mw) | H2O, | substitution | aq. |
| sample | cation | % M⁺ | cation | % M⁺⁺ | (Daltons) | (cPs) | (CmDs) | Soln.) |
| XA1517-041 | Na⁺ | 8.66 | Ca⁺⁺ | <0.01 | 1180000 | 1487 | 0.6-0.8 | 7.34 |
| XA1517-50-1 | Na⁺ | 6.47 | Ca⁺⁺ | 1.18 | 954000 | 1928 | 0.6-0.8 | 6.42 |
| XA1517-50-2 | Na⁺ | 4.32 | Ca⁺⁺ | 3.11 | 923000 | 1560 | 0.6-0.8 | 6.57 |
| XA1517-78 | Na⁺ | 0.10 | Ca⁺⁺ | 6.30 | 1050000 | 1324 | 0.6-0.8 | 6.20 |

### Example 4: Synthesis of Prototype Na⁺-Mg⁺⁺-CMC Polymer (XA1517-018)

63.0 grams of cellulose (42.00 grams of Gaomi 2600 and 21.00 grams of NB421) was combined in a 1.5 liter stainless steel reactor with 711.84 grams of isopropanol, 21.00 grams of deionized water, and 21.86 grams of magnesium chloride hexahydrate. The reactor was sealed, stirring was initiated using an overhead impellor, the reactor was cooled to 20° C., and the reactor was purged of oxygen by 3 vacuum/backfills with ultrapure nitrogen. Next, 95.39 grams of a 50% NaOH solution in deionized water was added followed by two more inert/backfills with ultrapure nitrogen. The mixture was allowed to stir at 20° C. for 1 hour before 88.90 grams of a 50% monochloroacetic solution in isopropanol was added. The reactor was then heated to 70° C. over the course of 30 minutes, held at 70° C. for 75 minutes, and cooled to 30° C. over the course of 30 minutes. The final material was then removed from the reactor, filtered, and the resulting wet cake was washed in 800 grams of 80/20 w/w methanol/water for 30 minutes and neutralized to a pH of 7 using concentrated hydrochloric acid. The slurry was then filtered, and the resulting wet cake was subjected to another wash and neutralization step. A third wash in the same solvent system was carried out, this time without neutralization and a final wash in 800 grams of neat methanol was carried out. The final material was dried at 65° C. using a fluid bed dryer until total moisture content of the final product was under 5 weight percent. The resulting, dried material was ground using a Retsch mill to the desired particle size.

### Example 5: Synthesis of Prototype Na⁺-Mg⁺⁺-CMC Polymer (XA1517-020)

63.0 grams of cellulose (42.00 grams of Gaomi 2600, and 21.00 grams of NB421) was combined in a 1.5 liter stainless steel reactor with 711.84 grams of isopropanol, and 49.68 grams of magnesium chloride hexahydrate. The reactor was sealed, stirring was initiated using an overhead impellor, the reactor was cooled to 20° C., and the reactor was purged of oxygen by 3 vacuum/backfills with ultrapure nitrogen. Next, 117.28 grams of a 50% NaOH solution in deionized water was added followed by two more inert/backfills with ultrapure nitrogen. The mixture was allowed to stir at 20° C. for 1 hour before 88.90 grams of a 50% monochloroacetic solution in isopropanol was added. The reactor was then heated to 70° C. over the course of 30 minutes, held at 70° C. for 75 minutes, and cooled to 30° C. over the course of 30 minutes. The final material was then removed from the reactor, filtered, and the resulting wet cake was washed in 800 grams of 80/20 w/w methanol/water for 30 minutes and neutralized to a pH of 7 using concentrated hydrochloric acid. The slurry was then filtered, and the resulting wet cake was subjected to another wash and neutralization step. A third wash in the same solvent system was carried out, this time without neutralization and a final wash in 800 grams of neat methanol was carried out. The final material was dried at 65° C. using a fluid bed dryer until total moisture content of the final product was under 5 weight percent. The resulting, dried material was ground using a Retsch mill to the desired particle size.

TABLE 2
Characteristic properties of Na⁺—Mg⁺⁺-CMC polymer prototypes

| Polymer | M⁺ | | M⁺⁺ | | | | | |
|---|---|---|---|---|---|---|---|---|
| sample | cation | % M⁺ | cation | % M⁺⁺ | MW | Viscosity | D.S. | pH |
| XA1517-018 | Na⁺ | 6.19 | Mg⁺⁺ | 1.08 | 1190000 | 1930 | 0.6-0.8 | 6.42 |
| XA1517-020 | Na⁺ | 5.25 | Mg⁺⁺ | 1.6 | 1180000 | 2047 | 0.6-0.8 | 6.57 |

D.S.—Degree of substitution (CmDs);
MW—Molecular Weight (Daltons),
Viscosity (cPs) (1% in water),
pH (@1% aqueous solution)

### Example 6: Synthesis of Prototype Na⁺—Zn⁺⁺-CMC Polymer (XA-1532-005)

73.00 grams of anhydrous zinc chloride was dissolved in 752.17 grams deionized water with stirring for 30 minutes. 758.09 grams of isopropanol was added with continual stirring. 120 grams of Aqualon™ CMC 7H3SXF grade carboxymethylcellulose (XA1517-041, Table 1) supplied by Ashland Specialty Ingredients were added to this solution with continual stirring, creating a heterogenous slurry. This mixture was stirred for 20 hours before being filtered to obtain a wet cake. The wet cake was dispersed in 1025 grams of 70/30 w/w methanol/water and stirred for 30 minutes before filtering again. This step was repeated two additional times and then a final wash in 1025 grams of neat methanol was carried out. The final material was dried at 65° C. using a fluid bed dryer until total moisture content of the final product was under 5 weight percent. Important polymer characteristics are shown in Table 3.

### Example 7: Synthesis of Prototype Na⁺—Zn⁺⁺-CMC Polymer (XA1532-068)

35.30 grams of anhydrous zinc chloride were dissolved in 681.36 grams deionized water with stirring for 30 minutes. 2779.66 grams of methanol were added with continual stirring. 275.00 grams of Aqualon™ CMC 7H3SXF grade carboxymethylcellulose (XA1517-041, Table 1) supplied by Ashland Specialty Ingredients were added to this solution with continual stirring, creating a heterogenous slurry. This mixture was stirred for 20 hours before being filtered to obtain a wet cake. The wet cake was dispersed in 2,350 grams of 70/30 w/w methanol/water and stirred for 30 minutes before filtering again. This step was repeated two additional times and then a final wash in 2,350 grams of neat methanol was carried out. The final material was dried at 65° C. using a fluid bed dryer until total moisture content of the final product was under 5 weight percent. Important polymer characteristics are shown in Table 3.

TABLE 3
Characteristic properties of Na⁺—Zn⁺⁺-CMC polymer prototypes

| | | | | | | | | |
|---|---|---|---|---|---|---|---|---|
| Polymer | M⁺ | | M⁺⁺ | | | | | |
| sample | cation | % M⁺ | cation | % M⁺⁺ | MW | Viscosity | D.S. | pH |

| | | | | | | |
|---|---|---|---|---|---|---|
| XA1532-005 | Na⁺ | 6.19 | Zn⁺⁺ | | | 0.75 |
| XA1532-068 | Na⁺ | 4.59 | Zn⁺⁺ | 4.84 | 1030000 | 0.76 |

D.S.—Degree of substitution (CmDs),
Mol. Wt (Mw) (Daltons)
Viscosity (1% in water) (cPs),
pH—(@1% aqueous solution)

### Example 8: Preparation of Denture Adhesive Formulations

General method of preparing standard denture adhesive creams is described herein. Components used and amount of the same utilized in formulating various denture adhesive formulations are described in Tables 4, 5, and 6. Denture adhesive creams were made in 25 gram batches. In a 100 ml plastic jar equipped with a screw cap, required amounts of petrolatum and mineral oil were charged in the plastic jar, melted by dipping the plastic jar in a water bath maintained at 70-80° C. and mixed well till a clear homogeneous liquid was obtained. Solid CMC polymer components were first added to the molten oil mix according to the amounts mentioned in Tables 4, 5, and 6, gently folded in and then homogenized by hand mixing. Mixed Ca/Na salt of poly(methyl vinyl ether/maleic acid)-poly (MVE/MA) (Gantrez™ MS-955, Ashland LLC.), was then added to the formulation according to the amounts mentioned in Tables 4, 5, and 6, gently folded in and then homogenized by hand mixing. The solid polymers were folded in, and hand blended with the oil mix to obtain a smooth paste, while maintaining the temperature of the mixture at 70-80° C. by dipping the plastic jar in a suitable temperature controlled water bath. Finally, the denture adhesive creams were subjected to high-speed bladeless centrifugal mixing in a Speed Mixer DAC 400 FVZ equipment from FlackTec Inc, Landrum, SC.

TABLE 4
Denture adhesive formulations with Na⁺—Mg⁺⁺-CMC

| | Components | | | | | | |
|---|---|---|---|---|---|---|---|
| | | | XA1517-041 | XA1517-018 | XA1517-020 | | |
| Formula | Petrolatum | Mineral | Na⁺- | Na⁺—Mg⁺⁺- | Na⁺—Mg⁺⁺- | Gantrez ™ | Total |
| # | (g) | oil (g) | CMC (g) | CMC (g) | CMC (g) | MS-955 (g) | (g) |
| Control | 5 | 5 | 6.75 | — | — | 8.25 | 25.0 |
| DA-29 | 5 | 5 | 5.06 | 1.69 | — | 8.25 | 25.0 |
| DA-30 | 5 | 5 | 3.38 | 3.38 | — | 8.25 | 25.0 |
| DA-31 | 5 | 5 | 1.69 | 5.06 | — | 8.25 | 25.0 |
| DA-32 | 5 | 5 | 5.06 | — | 1.69 | 8.25 | 25.0 |
| DA-33 | 5 | 5 | 3.38 | — | 3.38 | 8.25 | 25.0 |
| DA-34 | 5 | 5 | 1.69 | — | 5.06 | 8.25 | 25.0 |

TABLE 5
Denture adhesive formulations with Na⁺—Ca⁺⁺-CMC

| | Components | | | | | | | |
|---|---|---|---|---|---|---|---|---|
| | | | XA1517-041 | XA1517-50-1 | XA1517-50-2 | XA1517-078 | Gantrez ™ | |
| Formula | Petrolatum | Mineral | Na⁺- | Na⁺—Ca⁺⁺- | Na⁺—Ca⁺⁺- | Na⁺—Ca⁺⁺- | MS-955 | Total |
| # | (g) | oil (g) | CMC (g) | CMC (g) | CMC (g) | CMC (g) | (g) | (g) |
| DA-35 | 5 | 5 | 5.06 | 1.69 | — | — | 8.25 | 25.0 |
| DA-36 | 5 | 5 | 3.38 | 3.38 | — | — | 8.25 | 25.0 |
| DA-37 | 5 | 5 | 1.69 | 5.06 | — | — | 8.25 | 25.0 |
| DA-38 | 5 | 5 | 0.00 | 6.75 | — | — | 8.25 | 25.0 |
| DA-39 | 5 | 5 | 5.06 | — | 1.69 | — | 8.25 | 25.0 |
| DA-40 | 5 | 5 | 3.38 | — | 3.38 | — | 8.25 | 25.0 |
| DA-41 | 5 | 5 | 1.69 | — | 5.06 | — | 8.25 | 25.0 |
| DA-42 | 5 | 5 | 0.00 | — | 6.75 | — | 8.25 | 25.0 |
| DA-43 | 5 | 5 | 5.06 | — | — | 1.69 | 8.25 | 25.0 |
| DA-44 | 5 | 5 | 3.38 | — | — | 3.38 | 8.25 | 25.0 |
| DA-45 | 5 | 5 | 1.69 | — | — | 5.06 | 8.25 | 25.0 |
| DA-46 | 5 | 5 | 0.00 | — | — | 6.75 | 8.25 | 25.0 |

TABLE 6
Denture adhesive formulations with Na⁺—Zn⁺⁺-CMC

| | Components | | | | | | |
|---|---|---|---|---|---|---|---|
| | | | XA1517-041 | XA1532-005 | XA1517-068 | | |
| Formula | Petrolatum | Mineral | Na⁺- | Na⁺—Zn⁺⁺- | Na⁺—Zn⁺⁺- | Gantrez ™ | Total |
| # | (g) | oil (g) | CMC (g) | CMC (g) | CMC (g) | MS-955 (g) | (g) |
| DA-87 | 5 | 5 | 5.06 | 1.69 | — | 8.25 | 25.0 |
| DA-88 | 5 | 5 | 3.38 | 3.38 | — | 8.25 | 25.0 |
| DA-89 | 5 | 5 | 1.69 | 5.06 | — | 8.25 | 25.0 |
| DA-90 | 5 | 5 | 0.00 | 6.75 | — | 8.25 | 25.0 |
| DA-99 | 5 | 5 | 5.06 | — | 1.69 | 8.25 | 25.0 |
| DA-100 | 5 | 5 | 3.38 | — | 3.38 | 8.25 | 25.0 |
| DA-101 | 5 | 5 | 1.69 | — | 5.06 | 8.25 | 25.0 |
| DA-102 | 5 | 5 | 0.00 | — | 6.75 | 8.25 | 25.0 |

### Example 9: In-Vitro Denture Adhesive Test Method

Adhesion forces and relative denture adhesive film thickness were recorded on TA.XT Plus texture analyzer instruments from Stable Microsystems Texture Technologies Corp., equipped with a 50 Kg load cell and interfaced with a PC running Exponent software version 6.1.11.0. The texture analyzer instruments were equipped with a custom-built denture shaped plexiglass probe-fixture assembly. Artificial saliva infusion between the walls of the plexiglass probe-fixture assembly was achieved using peristaltic pumps. Real-time time-lapse images of denture adhesive creams undergoing adhesion performance evaluation on the texture analyzer instruments were recorded using a Canon EOS 5d Mark IV Digital SLR camera.

Prepared denture adhesive formulations were hand mixed to a homogeneous and uniform cream consistency before each test. Three nearly identical strips of denture adhesive cream were weighed out into the denture shaped cavity of the bottom plexiglass fixture on an analytical balance. Total mass of denture adhesive cream applied to the bottom fixture for each test was 2. 0 (+0.1) g. The bottom fixture was mounted on to the texture analyzer instrument and the top probe was moved down into the cavity of the bottom fixture to uniformly spread the denture adhesive cream and fill the bottom half of the cavity under a precise compression force of 4.5 Kg. Infusion of artificial saliva was initiated. For the entire duration of the experiment, the denture adhesive cream remained in contact and submerged under a thin level of artificial saliva which was continuously refreshed at a flow rate of 1 ml/minute. The 7-hour test sequence was initiated once the denture adhesive cream layer was fully covered with artificial saliva. Adhesion force (in Newtons) at instances of mastication and thickness of denture adhesive film between the top and the bottom probe (in mm) were continuously measured for the duration of the experiment. Each sample of prepared denture adhesive formulations was subjected to four consecutive experimental runs. The average plots from 4 runs for each sample were compared for adhesion performance, and thickness variation. Total adhesion for each sample was calculated as the area under the curve for the average plot of the 4 consecutive runs for each sample.

### Example 10: In-Vitro Adhesion Results

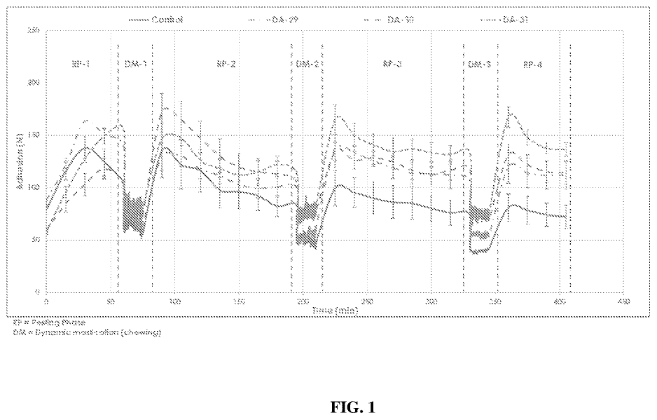

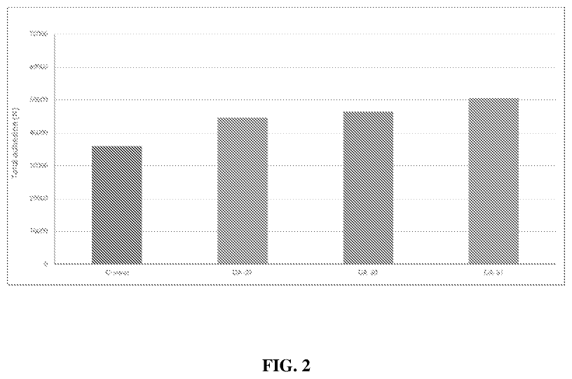

FIG. 1 represents overlay of average adhesion profiles of Control denture adhesive and denture adhesive formulae containing Na⁺-Mg⁺⁺-CMC (DA29-31). Adhesion profile trace of each denture adhesive sample is an average of four consecutive experimental runs. Each overlay plot was demarcated into alternating phases of slow chewing motion or Resting Phase (RP) and rapid chewing motion or Dynamic Mastication (DM). Adhesion forces were recorded and reported in Newton (N). FIG. 2 shows the Total Adhesion for each representative denture adhesive formulations (Control, DA29-31). Total Adhesion (in N) was measured as the total area under the curve for the average adhesion profile trace for each representative denture adhesive formulations.

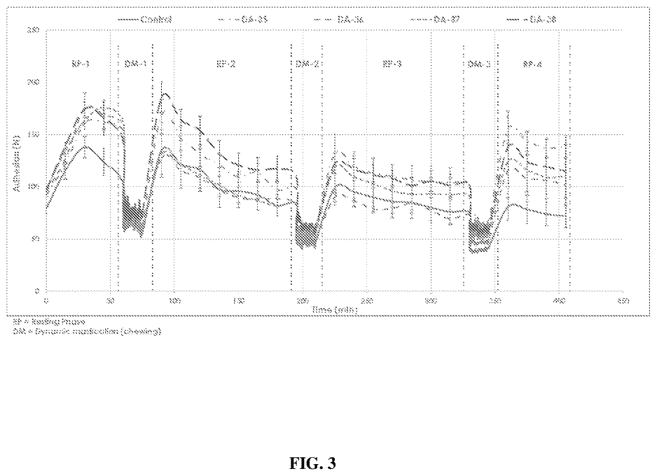

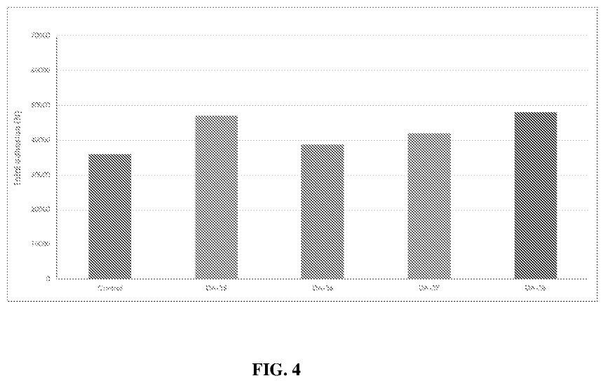

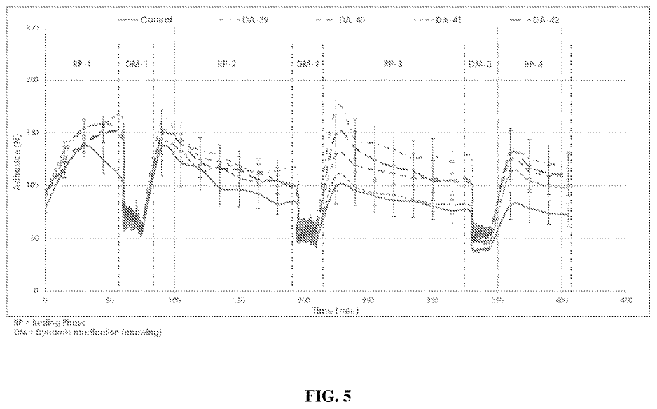

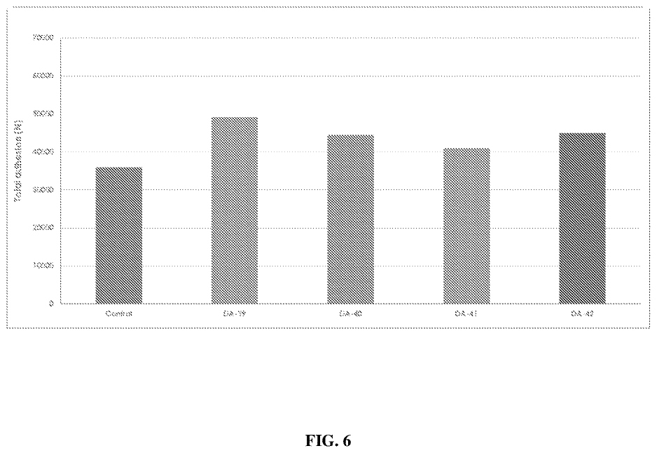

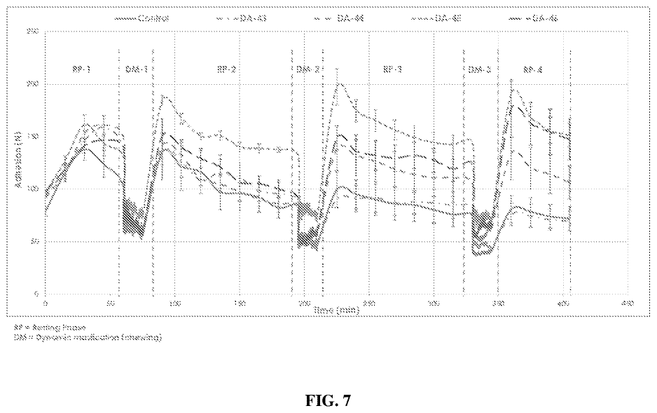

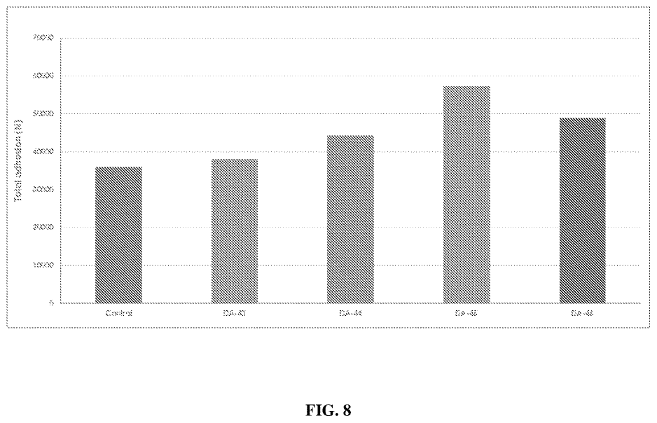

FIGS. 3, 5, and 7 represents overlay of average adhesion profiles of Control denture adhesive and denture adhesive formulae containing Na⁺—Ca⁺⁺-CMC (DA35-46). Adhesion profile trace of each denture adhesive sample is an average of four consecutive experimental runs. Each overlay plot was demarcated into alternating phases of slow chewing motion or Resting Phase (RP) and rapid chewing motion or Dynamic Mastication (DM). Adhesion forces were recorded and reported in Newton (N). FIGS. 4, 6, and 8 shows the Total Adhesion for each representative denture adhesive formulations (Control, DA35-46). Total Adhesion (in N) was measured as the total area under the curve for the average adhesion profile trace for each representative denture adhesive formulations.

FIGS. 9, and 11 represents overlay of average adhesion profiles of Control denture adhesive and denture adhesive formulae containing Na⁺—Zn⁺⁺-CMC (DA87-102). Adhesion profile trace of each denture adhesive sample is an average of four consecutive experimental runs. Each overlay plot was demarcated into alternating phases of slow chewing motion or Resting Phase (RP) and rapid chewing motion or Dynamic Mastication (DM). Adhesion forces were recorded and reported in Newton (N). FIGS. 10 and 12 show the Total Adhesion for each representative denture adhesive formulations (Control, DA87-102). Total Adhesion (in N) was measured as the total area under the curve for the average adhesion profile trace for each representative denture adhesive formulations. Total adhesion yielded by each prototype denture adhesive formulation tested by the in-vitro method are shown in Table 7.

TABLE 7
Denture adhesive total adhesion by in-vitro test method

| Denture adhesive | Total |
|---|---|
| sample designation | adhesion (N) |
| control | 35997.5 |
| DA-29 | 44636.3 |
| DA-30 | 46362.9 |
| DA-31 | 50474.9 |
| DA-35 | 47050.6 |
| DA-36 | 38731.3 |
| DA-37 | 41918.73 |
| DA-38 | 48008.16 |
| DA-39 | 49210.1 |
| DA-40 | 44545.7 |
| DA-41 | 41017.7 |
| DA-42 | 45008.4 |
| DA-43 | 38061.88 |
| DA-44 | 44278.48 |
| DA-45 | 57286.01 |
| DA-46 | 48878.49 |

DA—Denture Adhesive

### Example 11: Toothpaste Formula with Silica and Na⁺—Zn⁺⁺-CMC

A suitably sized vessel was charged with glycerin (1 wt. %-30 wt. %) and divalent cation functionalized Na⁺—Zn⁺⁺-CMC (0.1 wt. %-6 wt. %) was thoroughly dispersed in the glycerin with overhead stirrer. 70% solution of sorbitol (1 wt. %-30 wt. %) was then charged to the same vessel and mixed thoroughly with overhead stirrer. Deionized water (1 wt. %-40 wt. %) was then added to the vessel and mixed vigorously with overhead stirring till a homogeneous viscous gel-phase was obtained. A pre-made aqueous solution of sodium fluoride (0.1 wt. %-0.3 wt. %) and sodium saccharin (0.1 wt. %-0.5 wt. %) was added to the vessel and vigorously mixed till homogeneous. The gel-phase was transferred to a suitably sized receptor jar of a whip mixer, and abrasive silica Zeodent 113 (1 wt. %-20 wt. %) and thickening silica Zeodent® 165 (0 wt. %-10 wt. %) were added to the gel-phase and folded into the gel phase by hand mixing. Titanium dioxide (0.1 wt. %-2 wt. %) was added to the mixture and gently folded into the mixture by hand. The Mixture was then subjected to high speed mixing under vacuum with the whip mixer till a uniform paste was obtained. Finally, sodium lauryl sulfate surfactant (0.1 wt. %-5 wt. %) and flavor (0.1 wt. %-5 wt. %) was added to the paste, and subjected toa final round of high speed mixing under vacuum in the whip mixture to obtain complete toothpaste formula.

### Example 12: Toothpaste Formula with Calcium Carbonate and Na⁺—Zn⁺⁺-CMC

A suitably sized vessel was charged with 70% solution of sorbitol (1 wt. %-30 wt. %) and divalent cation functionalized Na⁺—Zn⁺⁺-CMC (0.1 wt. %-6 wt. %) was thoroughly dispersed in the sorbitol solution with overhead stirrer. Deionized water (1 wt. %-40 wt. %) was then added to the vessel and mixed vigorously with overhead stirring till a homogeneous viscous gel-phase was obtained. A pre-made aqueous solution of sodium monofluorophosphate (0.1 wt. %-2 wt. %), Trisodium phosphate decahydrate (0.1 wt. %-2 wt. %) and Sodium saccharin (0.1 wt. %-0.5 wt. %) was added to the vessel and vigorously mixed till homogeneous. The gel-phase was transferred to a suitably sized receptor jar of a whip mixer, and abrasive calcium carbonate (1 wt %-50 wt %) and thickening silica Zeodent® 165 (0 wt. %-10 wt. %) were added to the gel-phase and folded into the gel phase by hand mixing. Titanium dioxide (0.1 wt. %-2 wt. %) was added to the mixture and gently folded into the mixture by hand. The Mixture was then subjected to high speed mixing under vacuum with the whip mixer till a uniform paste was obtained. Finally, sodium lauryl sulfate surfactant (0.1 wt. %-5 wt. %) and flavor (0.1 wt. %-5 wt. %) was added to the paste and subjected to a final round of high speed mixing under vacuum in the whip mixture to obtain complete toothpaste formula.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. The embodiments of the invention in which an exclusive property or privilege is claimed are defined as follows.

What is claimed is:

1. A water-soluble salt of carboxylated polysaccharide functionalized with at least one mono-valent (M⁺) cation and at least one di-valent (M⁺⁺) cation.

2. The composition according to claim 1, wherein the functionalized carboxylated polysaccharide is selected from the group of carboxymethyl cellulose, carboxymethyl inulin carboxymethyl guar and carboxymethyl starch.

3. The composition according to claim 1, wherein the functionalized carboxylated polysaccharide is carboxymethyl cellulose.

4. A water-soluble salt of carboxymethyl cellulose (CMC) functionalized with at least one mono-valent (M⁺) cation and at least one di-valent (M⁺⁺) cation.

5. The water-soluble salt of carboxymethyl cellulose (CMC) according to claim 4, wherein the di-valent cation is present in an amount of about 0.2 to 7.5 wt. % of the total CMC.

6. The water-soluble salt of carboxymethyl cellulose (CMC) according to claim 4, wherein the mono-valent and di-valent cations are present in weight ratio of 75:1 to 1:75 in the final functionalized CMC polymer.

7. The water-soluble salt of carboxymethyl cellulose (CMC) according to claim 4, wherein the carboxymethyl cellulose (CMC) is optionally further combined with carboxymethyl cellulose (CMC) functionalized with at least one mono-valent (M⁺) cation.

8. The water-soluble salt of carboxymethyl cellulose (CMC) according to claim 4, wherein the functionalized carboxymethyl cellulose (CMC) has a degree of substitution in the range of 0.4 to 1.2.

9. The water-soluble salt of carboxymethyl cellulose (CMC) according to claim 4, wherein the functionalized carboxymethyl cellulose (CMC) has a degree of substitution in the range of 0.4 to 0.7.

10. The water-soluble salt of carboxymethyl cellulose (CMC) according to claim 4, wherein the functionalized carboxymethyl cellulose has a weight average molecular weight in the range of 2000 to 2000,000 Daltons.

11. The water-soluble salt of carboxymethyl cellulose (CMC) according to claim 4, wherein the functionalized carboxymethyl cellulose has a weight average molecular weight in the range of 80,000 to 1,300,000 Daltons.

12. The water-soluble salt of carboxymethyl cellulose (CMC) according to claim 4, wherein the functionalized carboxymethyl cellulose (CMC) maintained the inherent biodegradable nature.

13. The water-soluble salt of carboxymethyl cellulose (CMC) according to claim 4, wherein the functionalized carboxymethyl cellulose has a structure of (I): 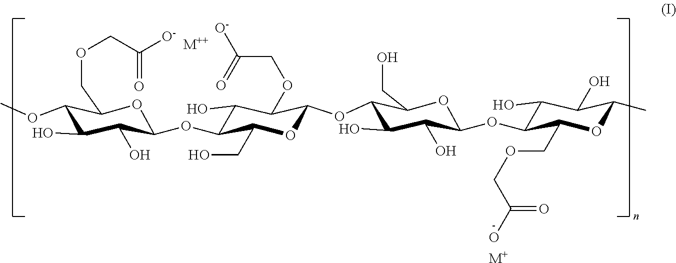
CMC functionalized with mono-valent cation and di-valent cation
wherein,
M⁺ is a mono-valent cation selected from the group consisting of sodium, potassium, and lithium; and
M⁺⁺ is a di-valent cation selected from the group consisting of calcium, magnesium, zinc, tin, and combinations thereof; and
wherein n≥2 to n≤2000, M⁺=0.1 to 10 wt. % and M⁺⁺=0.1 to 7.5 wt. % of the carboxymethyl cellulose.

14. The water-soluble salt of carboxymethyl cellulose (CMC) according to claim 4, wherein the functionalized carboxymethyl cellulose has a structure of (II): 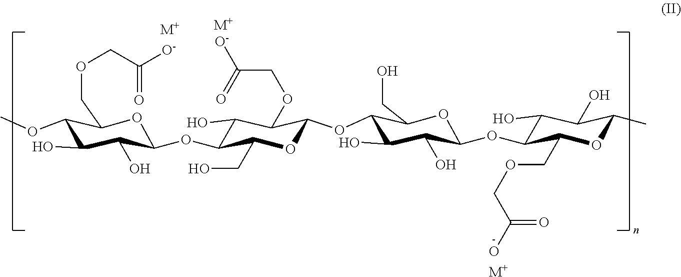
CMC functionalized with mono-valent cation
wherein,
M⁺ is a mono-valent cation selected from the group consisting of sodium, potassium, and lithium; and wherein n≥2 to n≤2000, M⁺=0.1 to 10 wt. % of the carboxymethyl cellulose.

15. The water-soluble carboxymethyl cellulose (CMC) according to claim 4, wherein the mono-valent (M⁺) cation is selected from the group consisting of sodium, potassium, and lithium.

16. The water-soluble carboxymethyl cellulose (CMC) according to claim 4, wherein the di-valent (M⁺⁺) cation is selected from the group consisting of calcium, magnesium, zinc, tin, and combinations thereof.

17. The water-soluble carboxymethyl cellulose (CMC) according to claim 4, wherein the functionalized carboxymethyl cellulose (CMC) is in dry powder form.

18. The water-soluble carboxymethyl cellulose (CMC) according to claim 4, wherein the functionalized carboxymethyl cellulose (CMC) is
(i) sodium-calcium carboxymethyl cellulose (Na⁺—Ca⁺⁺-CMC),
(ii) sodium-magnesium carboxymethyl cellulose (Na⁺-Mg⁺⁺-CMC),
(iii) sodium-zinc carboxymethyl cellulose (Na⁺—Zn⁺⁺-CMC),
(iv) sodium-calcium-zinc carboxymethyl cellulose (Na⁺—Ca⁺⁺—Zn⁺⁺-CMC),
(v) sodium-calcium-magnesium carboxymethyl cellulose (Na⁺-Ca⁺⁺-Mg⁺⁺-CMC),
(vi) sodium-magnesium-zinc carboxymethyl cellulose (Na⁺-Mg⁺⁺—Zn⁺⁺-CMC),
(vii) sodium-tin-carboxymethyl cellulose (Na⁺—Sn⁺⁺-CMC),
(viii) sodium-calcium-tin-carboxymethyl cellulose (Na⁺—Ca⁺⁺—Sn⁺⁺-CMC),
(ix) sodium-magnesium-tin-carboxymethyl cellulose (Na⁺-Mg⁺⁺—Sn⁺⁺-CMC), or
(x) sodium-zinc-tin-carboxymethyl cellulose (Na⁺—Zn⁺⁺—Sn⁺⁺-CMC).

19. The water-soluble carboxymethyl cellulose (CMC) according to claim 6, wherein the functionalized carboxymethyl cellulose (CMC) is a combination of sodium carboxymethyl cellulose with at least one functionalized carboxymethyl cellulose (CMC) selected from compounds of claim 18 (i), to (x); or combinations thereof.

20. The water-soluble carboxymethyl cellulose (CMC) according to claim 6, wherein the functionalized carboxymethyl cellulose (CMC) is sodium-calcium-carboxymethyl cellulose (Na⁺—Ca⁺⁺-CMC) or sodium-magnesium-carboxymethyl cellulose (Na⁺-Mg⁺⁺-CMC) or sodium-zinc-carboxymethyl cellulose (Na⁺—Zn⁺⁺-CMC).

21. The water-soluble carboxymethyl cellulose (CMC) according to claim 6, wherein the functionalized carboxymethyl cellulose (CMC) is a combination of (i) sodium carboxymethyl cellulose (Na⁺-CMC) with (ii) (a) sodium-calcium carboxymethyl cellulose (Na⁺—Ca⁺⁺-CMC) or (b) sodium-magnesium-carboxymethyl cellulose (Na⁺-Mg⁺⁺-CMC) or (c) sodium-zinc-carboxy methyl cellulose (Na⁺—Zn⁺⁺-CMC).

22. The water-soluble carboxymethyl cellulose (CMC) according to claim 4, wherein the functionalized carboxymethyl cellulose (CMC) is used in oral care composition, pharmaceutical composition, home care composition, industrial composition, skin care composition, food and nutraceutical composition, coating composition, and oil & energy composition.

23. The water-soluble carboxymethyl cellulose (CMC) according to claim 4, wherein the functionalized carboxymethyl cellulose (CMC) is used in oral care composition selected from mucoadhesive composition, toothpaste/dentrifice composition, oral therapeutic composition, mouth rinse composition and denture adhesive composition.

24. A mucoadhesive composition comprising:
(i) (a) 0.1 wt. % to 60 wt. % of water soluble salt of carboxymethyl cellulose (CMC) of claim 4; or
(b) 0.1 wt. % to 60 wt. % of a combination of water soluble salt of carboxymethyl cellulose (CMC) of claim 4, and carboxymethyl cellulose (CMC) functionalized with at least one mono-valent (M⁺) cation;
(ii) 0.1 wt. % to 60 wt. % of a maleic acid or maleic anhydride co-polymer of C₁ to C₄ alkyl vinyl ether and maleic acid or anhydride monomers in substantially alternating monomer structure and having 50 to 90 wt. % of the acid moieties in the polymer converted to a metal salt(s) or mixed salts; and
(iii) 0.1 wt. % to 80 wt. % of at least one additional ingredient.

25. The mucoadhesive composition according to claim 24 (i), wherein the water soluble functionalized carboxymethyl cellulose (CMC) has a degree of substitution in the range of 0.4 to 1.2.

26. The mucoadhesive composition according to claim 24 (i), wherein the water soluble functionalized carboxymethyl cellulose (CMC) has a degree of substitution in the range of 0.4 to 0.7.

27. The mucoadhesive composition according to claim according to claim 24, wherein the functionalized carboxymethyl cellulose has a weight average molecular weight in the range of 2000 to 1,500,000 Daltons.

28. The mucoadhesive composition according to claim 24, wherein the maleic acid or maleic anhydride co-polymer of C1 to C4 alkyl vinyl ether and maleic acid or anhydride monomers in substantially alternating monomer structure and having 50 to 90 wt. % of the acid moieties in the polymer converted to a salt or mixed salts of at least one mono-valent cation (M⁺) and at least one di-valent cation (M⁺⁺).

29. The mucoadhesive composition according to claim 28, wherein the maleic acid or maleic anhydride copolymer comprises maleic acid or maleic anhydride and a C1-C4 alkyl vinyl ether, having a predetermined weight average molecular weight of about 500,000 to 3,000,000 made by copolymerizing about 50 mole percent of maleic acid or maleic anhydride, about 50 mole percent of a C1-C4 alkyl vinyl ether, in the presence of a free radical initiator, at about 50° to 150° C. in a solvent or solvent free process to produce a uniform, fine powder having substantially no residual maleic anhydride.

30. The mucoadhesive composition according to claim 28, wherein the maleic acid or maleic anhydride and a C1-C4 alkyl vinyl ether is a methyl vinyl ether-maleic acid copolymer or methyl vinyl ether-maleic anhydride copolymer has a number average molecular weight of between about 50,000 and about 500,000 wherein about 50 to about 100 wt. % of the carboxyl units in the polymer are converted to a mixture of metal salts selected from the group consisting of calcium, sodium, strontium, zinc, magnesium, iron, boron, aluminum, potassium, vanadium, chromium, manganese, nickel, copper, yttrium, titanium, and mixtures thereof.

31. The mucoadhesive composition according to claim 29, wherein the salts of the copolymer of methyl vinyl ether-maleic acid or methyl vinyl ether-maleic anhydride has a specific viscosity of from about 2.5 to 5.0 when measured as a 1% w/v solution in methyl ethyl ketone (MEK) solution at 25° C.

32. The mucoadhesive composition according to claim 29, wherein the copolymer of methyl vinyl ether-maleic acid or methyl vinyl ether-maleic anhydride or salts thereof comprises from about 18 to about 66 wt. % of said composition.

33. The mucoadhesive composition according to claim 29, wherein the methyl vinyl ether-maleic acid or methyl vinyl ether-anhydride copolymer has a weight average molecular weight of from about 700,000 to about 3,000,000.

34. The mucoadhesive composition according to claim 24, wherein the additional ingredient is selected from the group consisting of abrasives, anti-caking agents, anti-fungal agents, anti-microbial agents, anesthetic agents, antioxidants, anti-biotics, anti-inflammatory agents, binders, buffers, colors, cooling agents, dentinal desensitizing agents, dispersants, enzymes, foaming agents, flavors, fillers, fragrances, gelling agents, humectants, hydrophobic carriers, hydrophilic non-oil components, preservatives, pigments, plasticizers, pain relieving agents, sweeteners, thickening agents, viscosity modifiers, vehicles, surfactants, stabilizers, sensates and mixtures thereof.

35. The mucoadhesive composition according to claim 24, wherein the additional ingredient is selected from petrolatum, mineral oil, olive oil, vegetable oil, silicon, glycerin, polyethylene glycol, propylene glycol, polypropylene glycol, poly(ethylene oxide-propylene oxide) copolymer, diethylene glycol, triethylene glycol, sorbitol, water, and mixtures thereof.

36. The mucoadhesive composition according to claim 24, wherein the mucoadhesive composition is a buccal composition, an oral composition, a vaginal composition, a nasal composition, a rectal composition, an ocular composition, a sublingual composition, a palatal composition, or a denture adhesive composition.

37. The mucoadhesive composition according to claim 24, wherein the mucoadhesive composition is formulated into an ointment, a lotion, a liquid, a solid, a semisolid, an emulsion, a powder, a dispersion, a gel, a patch, an aerosol or a spray.

38. A denture adhesive composition comprising:
(i) (a) 0.1 wt. % to 60 wt. % of water soluble salt of carboxymethyl cellulose (CMC) of claim 4; or
(b) 0.1 wt. % to 60 wt. % of a combination of water soluble salt of carboxymethyl cellulose (CMC) of claim 4, and carboxymethyl cellulose (CMC) functionalized with at least one mono-valent (M⁺) cation;
(ii) 0.1 wt. % to 60 wt. % of a maleic acid or maleic anhydride co-polymer of C₁ to C₄ alkyl vinyl ether and maleic acid or anhydride monomers in substantially alternating monomer structure and having 50 to 90 wt. % of the acid moieties in the polymer converted to a metal salt(s) or mixed salts; and
(iii) 0.1 wt. % to 80 wt. % of at least one additional ingredient.

39. The denture adhesive composition according to claim 38, wherein the water soluble functionalized carboxymethyl cellulose (CMC) has a degree of substitution in the range of 0.4 to 0.7.

40. The denture adhesive composition according to claim 38, wherein the water soluble functionalized carboxymethyl cellulose (CMC) has a degree of substitution in the range of 0.4 to 1.2.

41. The denture adhesive composition according to claim according to claim 38, wherein the functionalized carboxymethyl cellulose has a weight average molecular weight in the range of 2000 to 1,500,000 Daltons.

42. The denture adhesive composition according to claim 38, wherein the maleic acid or maleic anhydride co-polymer of C₁ to C₄ alkyl vinyl ether and maleic acid or anhydride monomers is mixed sodium and calcium salt of meth vyl vinyl ether and maleic anhydride having a specific viscosity of from about 2.5 to 5.0 when measured as a 1% w/v solution in methyl ethyl ketone (MEK) solution at 25° C.

43. A toothpaste composition comprising:
(i) (a) 0.1 wt. % to 20 wt. % of water soluble salt of carboxymethyl cellulose (CMC) of claim 4; or
(b) 0.1 wt. % to 20 wt. % of a combination of water soluble salt of carboxymethyl cellulose (CMC) of claim 4, and carboxymethyl cellulose (CMC) functionalized with at least one mono-valent (M⁺) cation;
(ii) 0.1 to 60 wt. % of an orally acceptable solvent or carrier selected from water, glycerin, sorbitol, propylene glycol, polypropylene glycol, PEG, ethylene oxide-propylene oxide polymers or combinations thereof;
(iii) 0.1 wt. % to 20 wt. % of orally acceptable polymers of monomers including but not limited to maleic anhydride, maleic acid, C₁ to C₄ alkyl vinyl ethers, alkylene oxides, alkyl lactams, triglycerides, animal or vegetable oils, or combinations thereof, and
(iv) 0.1 wt. % to 80 wt. % of at least one additional ingredient selected from the group consisting of abrasives, anti-caking agents, actives like fluoride providing compounds, anti-fungal agents, anti-microbial agents, anesthetic agents, antioxidants, anti-plaque agents, anti-tartar agents, anti-biofilm agents, anti-sensitivity agents, anti-gingivitis agents, anti-biotics, anti-inflammatory agents, binders, buffers, colorants, cooling agents, deposition agents, desensitizing agents, dispersants, encapsulants, enzymes, foaming agents, flavors, fillers, fragrances, film forming polymers, gelling agent, humectants, hydrophobic carriers, hydrophilic non-oil components, mouth feel agents, minerals, preservatives, peroxides, salts, pigments, pH modifiers, plasticizers, pain relieving agents, rheology modifiers, sweeteners, surfactants, stabilizers, sensates, strain removal agents, stain prevention agents, thickening agents, viscosity modifiers, vehicles, vitamins whitening agents, vegetable and animal oils, or combinations thereof, zinc salts of organic and inorganic acids, or combinations thereof.

44. The toothpaste composition according to claim 43, wherein
(i) the thickening agent is selected from the group consisting of:
(a) water-soluble cellulose ethers such as sodium carboxymethylcellulose, sodium carboxymethyl hydroxyethyl cellulose and hydroxyethyl cellulose,
(b) vinylpyrrolidone polymers such as N-vinylpyrrolidone, N-vinyl-2-pyrrolidone, N-vinyl-2-pyrrolidinone, and polyvinyl pyrrolidone,
(c) carboxy vinyl polymers (carbomers),
(d) carrageenan, laponite and natural gums such as gum karaya, xanthan gum, guar gum, gum arabic, and gum tragacanth,
(e) colloidal magnesium aluminum silicate, hydrated silica, thickening silica or finely divided silica, and
(f) homopolymers of acrylic acid cross-linked with an alkyl ether of pentaerythritol or an alkyl ether of sucrose; and
(ii) the abrasive is selected from the group consisting of silica, calcium carbonate, calcium bicarbonate, sodium carbonate and sodium bicarbonate.

45. A method for preparing mono-valent-di-valent (M⁺-M⁺⁺)-carboxymethyl cellulose, the method comprising:
(i) preparing an aqueous solution of (a) M⁺⁺ chloride, M⁺⁺ sulfate or M⁺⁺ carbonate and (b) C2-C4 alcohol;
(ii) adding sodium (M⁺)-carboxymethyl cellulose to step (i) and stirring the resultant for at least 30 minutes at an ambient temperature;
(iii) filtering the resultant of step (ii) to obtain wet cake;
(iv) stirring the wet cake of step (iii) in a hydroalcoholic solution for at least 30 minutes;
(v) repeating the step (iv) for at least 3 times to obtain pure wet cake of M⁺-M⁺⁺-carboxymethyl cellulose;
(vi) drying the wet cake of step (v) on a fluid-bed dryer for at least 15-30 minutes at 50-70° C. to obtain dry M⁺-M⁺⁺-carboxymethyl cellulose; and
(vii) grinding the resultant of step (vi) to isolate dry powder of M⁺-M⁺⁺-carboxymethyl cellulose.

46. The method according to claim 45, wherein the M⁺-M⁺⁺-carboxymethyl cellulose is sodium-calcium carboxymethyl cellulose.

47. The method according to claim 45, wherein the M⁺⁺ chloride, M⁺⁺ sulfate or M⁺⁺ carbonate is calcium chloride, calcium sulfate or calcium carbonate.

48. The method according to claim 45, wherein the C1-C4 alcohol is selected from methanol, ethanol, propanol, isopropanol, and butanol.

49. The method according to claim 45, wherein the grinding is carried out in a mill using 0.75, 0.20 and 0.08 mm screens.

50. The method according to claim 45, wherein the dry powder has a mean particle diameter in the range of 20 to 300 microns.
